(19) United States
(12) Patent Application Publication
(10) Pub. No.: US 2013/0196336 A1
(43) Pub. Date: Aug. 1, 2013

(54) TRANSGLUTAMINASE 6 AS A DIAGNOSTIC INDICATOR OF AUTOIMMUNE DISEASES

(71) Applicants: Marios Hadjivassilliou, Sheffied (GB); Daniel Aeschlimann, Dinas Powys (GB)

(72) Inventors: Marios Hadjivassilliou, Sheffied (GB); Daniel Aeschlimann, Dinas Powys (GB)

(21) Appl. No.: 12/594,926

(22) PCT Filed: Apr. 7, 2008
(86) PCT No.: PCT/EP08/02744

§ 371 Date: Feb. 8, 2010

Related U.S. Application Data
(60) Provisional application No. 60/924,567, filed on May 21, 2007.

(30) Foreign Application Priority Data
Apr. 6, 2007 (EP) 07007262.4

(51) Int. Cl. G01N 33/68; G01N 33/86; C12N 9/10

(52) U.S. Cl. CPC G01N 33/6896; G01N 33/6854; C12N 9/1044; G01N 33/86
USPC 435/7.1; 435/193

(57) ABSTRACT

The embodiments relate to the diagnosis of disorders or dysfunctions characterised by autoimmune responses to a novel antigen, transglutaminase 6, by the detection of autoantibodies to the novel antigen.

### BACKGROUND OF THE INVENTION

1. Field of the Invention

The present invention relates to the diagnosis of disorders or dysfunctions characterised by autoimmune responses to a novel antigen, transglutaminase 6, by the detection of autoantibodies to the novel antigen.

2. Description of the Relevant Art

Transglutaminases are a family of structurally and functionally related enzymes that post-translationally modify proteins by catalyzing a Ca²⁺-dependent transferase reaction between the γ-carboxamide group of a peptide-bound glutamine residue and various primary amines. Most commonly, intra- or intermolecular γ-glutamyl-ε-lysine crosslinks are formed by reaction with the 6-amino group of a lysine residue. The action of these enzymes consequently results in the formation of covalently crosslinked, often insoluble supramolecular structures and has a well established role in tissue homeostasis in many biological systems. Nine different transglutaminase genes have been characterised in higher vertebrates on the basis of their primary structure. The respective gene products can be found throughout the body; however, each enzyme isoform is characterised by its own unique tissue distribution, whereby each may be present in a number of different tissues, often in combination with other transglutaminase isoforms.

However, in the absence of suitable amines for crosslinking, transglutaminases hydrolyze peptide-bound glutamine to glutamate (by reaction with H₂O) (Mycek and Waelsch,1960. J. Biol. Chem. 235: 3513-3517), the biological significance of which has only recently been established in connection with coeliac disease (Molberg et al., 1998. Nature Med. 4: 713-717; Van de Wal et al., 1998. J. Immunol. 161: 1585-1588). Coeliac disease is a gluten sensitivity enteropathy that is linked to the consumption of certain cereal proteins called prolamines. Wheat protein, called gluten, contains the prolamine gliadin, which serves as a model for scientific research on coeliac disease. Coeliac disease is a common immune mediated, chronic inflammatory disorder, the aetiology of which is only partially understood. Prevalence studies looking at the incidence in the healthy population suggest that as much as 1% of the population in Western Europe and the US may be affected but only 1 in 8 of those present with intestinal disease. Undetected/untreated coeliac disease is associated with a significant risk of gastrointestinal malignancies, osteoporosis, and a higher risk of developing other autoimmune diseases such as diabetes, thyroid and liver disease. Based on this, there is a strong case for screening for silent coeliac disease and preventative dietary restrictions irrespective of the presence of symptoms and/or associated diseases.

Coeliac disease is a T-cell-mediated autoimmune disorder characterized by its close linkage to specific human lymphocyte antigen alleles: HLA-DQ2 (95%) and HLA-DQ8 (remaining 5%). In susceptible individuals, consumption of gluten triggers a CD4⁺ T-cell response to gliadin as well as a B-cell response to gliadin and self antigens. Transglutaminase 2 (TG2) has been identified as the autoantigen that is recognized in the endomysium of the gut by sera from coeliac disease patients (Dieterich W. et al., 1997. Nat. Med. 3: 797-801). Activation of gliadin specific CD4+ T-cells by DQ2+ Cd11c+ dendritic cells in the small intestinal mucosa produces proinflammatory cytokines which trigger the organ specific inflammatory reaction. Hallmark symptoms include weight loss, abdominal bloating and pain, and diarrhoea as a consequence of the mucosal lymphocyte infiltration-triggered inflammation in the small bowel which may ultimately cause complete villous atrophy. Characteristically, the condition improves upon gluten exclusion from the diet.

The Gold standard for the diagnosis of coeliac disease is still the histological examination of intestinal biopsies. Whilst the triad of villous atrophy, crypt hyperplasia and increase in the intraepithelial lymphocytes is what defines coeliac disease, more subtle morphological changes depending on the gluten load have subsequently been proposed by Marsh and are now widely accepted (Marsh classification). Since TG2 was discovered as the autoantigen of coeliac disease, an ELISA-based detection for TG2 autoantibodies has been made commercially available and is used in coeliac disease diagnosis and for monitoring the effectiveness of therapy.

The fact that TG2 is the predominant autoantigen also implicates the enzyme in the pathogenic process. TG2 is thought to contribute to the development of the disease in susceptible individuals in at least two independent ways: Firstly, by deamidating gluten peptides and thereby increasing their reactivity with HLA-DQ2/DQ8 which potentiates the T-cell response (Molberg et al., 1998. Nature Med. 4: 713-717; Van de Wal et al., 1998. J. lmmunol. 161: 1585-1588; Fleckenstein et al., 2002. J. Biol. Chem. 277: 34109-34116) and secondly, by haptenisation of self-antigens through crosslinking with gliadins (Fleckenstein et al., 2004. J. Biol. Chem. 279: 17607-17616; Dietrich et al., 2006. Gut 55: 478-484). The absence of intestinal T-cell responses to gluten in healthy individuals and the preferential T-cell responses to deamidated gluten fragments in patients with coeliac disease indicates that there is tolerance to unmodified gluten peptides. Therefore, the deamidation of gluten peptides, catalysed by TG2, may be central to the disruption of tolerance and disease development. The mechanism underlying the formation of autoantibodies in coeliac disease is not understood. The production of the anti-TG2 IgA is likely dependent on cognate T-cell help to facilitate isotype switching of autoreactive B-cells. Autoreactive T-cells to TG2 are unlikely to survive thymic selection, and to our knowledge, have thus far not been isolated from coeliac disease patients. Formation of complexes of gluten and TG2 may permit gluten reactive T-cells to provide the necessary help to TG2-specific B-cells. This model is attractive as it also explains why serum TG2 antibodies disappear when patients are subjected to a gluten free diet, i.e. when gluten disappears, so does the T-cell help needed to drive the B-cell response.

While coeliac disease is believed to primarily affect the small bowel, it has been reported that gluten sensitivity may also manifest or be associated with other conditions including anaemia, osteoporosis, type 1 diabetes with poor glycaemic control, autoimmune thyroiditis, liver disease (especially autoimmune hepatitis, non-alcoholic steatohepatitis, primary biliary cirrhosis, primary sclerosing cholangitis), infertility and recurrent miscarriage, colitis (especially microscopic lymphocytic colitis), IgA deficiency, and recurrent mouth ulcers. Lymphomas, mostly T-cell type, and other malignant tumours, particularly carcinoma of the small bowel, less frequently of stomach and oesophagus, are also associated with coeliac disease. Loss of response to a gluten free diet (refractory coeliac disease) and ulcerative jejunitis are two recently described complications of coeliac disease that may progress to an enteropathy-associated T-cell lymphoma.

An association of coeliac disease with various autoimmune diseases has been suggested and it has been reported that autoimmune diseases are 3 to 10 times more frequent in coeliac disease patients than in the general population possibly indicating a more generalised autoimmune phenomenon (WO 01/01133; Zelnik et al., 2004. Pediatrics 113: 1672-1676; Fasano 2006. Curr. Opin. Gastroenterol. 22: 674-679). Autoimmune conditions that are well established to be associated with coeliac disease include: type 1 diabetes, autoimmune thyroid diseases (Graves' disease and Hashimoto's thyroiditis), Sjögren's syndrome, autoimmune liver disease including primary biliary cirrhosis and sclerosing cholangitis, IgA nephropathy or IgA glomerulonephritis. Other conditions reported to be associated with coeliac disease include autoimmune haemolytic anaemia and thrombocytopenic purpura, cardiomyopathy, recurrent pericarditis, polymyositis, inclusion body myositis, partial lipodystrophy, relapsing polychondritis, rheumatoid arthritis, SLE (splenic atrophy), Addisons's disease, ulcerative colitis, atrophic gastritis-pernicious anaemia, vasculitis (both systemic and cutaneous), sarcoidosis and vitiligo. However, a recent study demonstrated that there was no correlation between autoimmune conditions independent of gluten sensitive disease and autoantibodies to TG2 (Sardy et al., 2007. Clin. Chim. Acta 376: 126-135).

It is a matter of debate whether the autoantibodies to TG2 are involved in the development of extraintestinal manifestations of coeliac disease. Evidence in support of this comes from the observation that in some instances ectopic manifestation of the disease is associated with the formation of IgA deposits in the respective tissue (Korponay-Szabo et al. 2004. Gut 53: 641-648). Also, osteoporosis has been demonstrated in clinically silent (no evidence of malabsorption) coeliac disease (Mustalahti 1999. Lancet 354: 744-745; Sugai et al 2002. J. Clin. Immunol. 22: 353-362) suggesting that an immune response to TG2 can affect bone homeostasis independent of intestinal pathology. Autoantibody production is alleviated by a gluten free diet which results in a concurrent increase in bone mineral density (Sugai et al 2002. J. Clin. lmmunol. 22: 353-362) or can reverse acute liver disease (Kaukinen et al., 2002. Gastroenterology 122:881-888), consistent with a strict correlation between TG2 autoantibodies and gluten sensitivity. However, recent research has indicated that the expression of cereal protein sensitivity as a dermatopathy (dermatitis herpetiformis) rather than an enteropathy may be determined by autoimmunity directed towards epidermal transglutaminase (TG3) rather than TG2 (WO 01/01133). Dermatitis herpetiformis is the skin manifestation of cereal protein sensitivity and is characterised by an itchy vesicular rash (typically located over the extensor surfaces of the major joints) and granular IgA deposits in the papillary dermis. The hypothesis for the aetiology of this pathology is that the autoantibodies generated initially by these patients cross-react between TG2 and TG3, as there is no evidence suggesting that TG3 is expressed in human intestine (Sárdy et al., 2002. J Exp Med 195:747-757). Prolonged gluten challenge results subsequently in the development of antibodies preferentially reacting with TG3 in these patients. However, two basic observations suggest that this interpretation is likely too simplistic. Firstly, besides TG3 several other highly homologous transglutaminase isozymes including TG5, TG6 and TG7 are expressed in epithelial tissues—TG5 for example is present in the jejunum—and the likelihood of generating cross reactive antibodies should be similar, yet only antibodies to TG3 co-localize with the characteristic IgA deposits in the papillary dermis (Sardy et al., 2002. J Exp Med 195:747-757). It is also surprising that the IgA deposits accumulate in a local where normally TG3 is absent indicating that they may originate from immune complexes formed in the circulation. Secondly, autoantibodies to TG3 are frequently observed in coeliac disease patients without apparent skin involvement (Example 3). Thus, it appears that while autoantibodies to TG2 are an excellent indicator of cereal protein sensitivity disease it is unclear whether other transglutaminases are involved in the pathological process and may therefore also be useful in the diagnosis of autoimmune disorders.

It has previously been demonstrated that TG2 can be used to diagnose autoimmune disorders including coeliac disease and sprue. US 2002/0076834 discloses the diagnosis of coeliac disease and sprue by the detection of autoantibodies to TG2. WO 01/01133 discloses the diagnosis of autoimmune disease of the gluten sensitive enteropathy type or of autoimmune diseases associated with gluten sensitive enteropathy by detection of autoantibodies to TG2.

Recently, the present inventors identified a novel transglutaminase gene, TGM6 (SEQ ID No. 1), which encodes the enzyme transglutaminase 6/transglutaminase y (TG6, SEQ ID No. 2) (WO 02/22830) which the inventors have since shown to constitute a functional transamidase. RT-PCR analysis of a large number of different human cell lines and tissues revealed a very restricted expression pattern of TG6 whereby expression could be identified only in a lung small cell carcinoma cell line (H69).

### SUMMARY OF THE INVENTION

It has been surprisingly found that the use of TG6 or an antigenically active fragment thereof, for detection of autoantibodies reacting with transglutaminase 6 is useful for the diagnosis of an autoimmune disorder.

In an embodiment, transglutaminase 6, or an antigenically active fragment thereof, is used for the detection of autoantibodies reacting with transglutaminase 6 for the diagnosis of an autoimmune disorder characterized by the presence of autoantibodies reacting with transglutaminase 6, wherein the autoimmune disorder is a neurological disorder or is characterized by neurological dysfunction

The object of the present invention is solved by the teaching of the independent claims. Further advantageous features, aspects and details of the invention are evident from the dependent claims, the description, and the examples of the present application.

### DESCRIPTION OF THE FIGURES

Advantages of the present invention will become apparent to those skilled in the art with the benefit of the following detailed description of embodiments and upon reference to the accompanying drawings in which:

FIG. 1 depicts an analysis of TG6 expressing cells isolated from cerebral cortex of newborn mice for cell type specific markers by flow cytometry. TG6 positive cells formed two clusters marked area R1 and R2;

FIG. 2 depicts a plot of the TG6 positive cells gated in area R1 (left panel) and R2 (right panel) as shown in FIG. 1 in relation to the expression of the markers for distinct cell populations: β-tubulin III isoform for neurons (FIG. 2A), glial fibrillary acidic protein (GFAP) for astrocytes (FIG. 2B) and RIP for oligodendrocytes (FIG. 2C). Controls with non-specific Ig of relevant species are given in FIG. 2D;

FIG. 3 depicts an SDS-PAGE analysis of purified recombinant transglutaminases. Proteins were separated in 4-20% SDS-PAGE gels under reducing conditions and stained with Coomassie brilliant blue R. A, recombinant human TG2 after the initial Ni²⁺-chelating affinity chromatography step (lane 2) and 1 μg (lane 3), 4 μg (lane 4) or 20 μg (lane 5) of the final product; B, purified recombinant human TG3 (lane 2, 0.2 μg; lane 3, 1 μg; lane 4, 5 μg); and C, purified recombinant human TG6 (lane 2, 0.2 μg; lane 3, 1 μg; lane 4, 5 μg). Mol wt standards were applied to lane 1 of each gel;

FIG. 4 depicts an analysis of serum anti-TG2 IgA (A), anti-TG6 IgA (B), anti-TG3 IgA (C). N, healthy blood donor; GA, gluten ataxia; GAE, gluten ataxia with enteropathy; CD, untreated coeliac disease; GenA, genetic ataxia; SMS, stiff person syndrome; Pneo, paraneoplastic syndromes; Misc others, unrelated neurological condition; GN, gluten sensitive peripheral neuropathy;

FIG. 5 depicts an analysis of serum anti-TG2 IgG (A), anti-TG6 IgG (B), anti-TG3 IgG. Note, 1 GAE patient is displayed as the maximum (140AU) but the reading exceeded the range (227AU). N, healthy blood donor; GA, gluten ataxia; GAE, gluten ataxia with enteropathy; CD, untreated coeliac disease; GenA, genetic ataxia; SMS, stiff person syndrome; Pneo, paraneoplastic syndromes; Misc others, unrelated neurological condition; GN, gluten sensitive peripheral neuropathy;

FIG. 6 depicts the effect of preincubation of sera with selected antigens on the antibody reaction measured by ELISA. A, TG2 IgA and IgG ELISA results for an example of a serum sample from a blood donor (normal serum 2) and from a coeliac disease patient (CD1) with or without incubation with 10 or 50 μg/ml TG2 and relevant controls. B, TG2 IgA and TG6 IgA ELISA results for a serum sample of the blood donor (normal serum 2), coeliac disease patient (CD1) and a gluten ataxia patient (GA16) (left panel) and TG2 IgA ELISA results for the same ataxia patient serum after preincubation with either TG2 or TG6; and

FIG. 7 depicts remaining IgA reactivity of sera from patients with CD or gluten ataxia after preincubation with TG2 or TG6. Representative examples of individual sera are shown in panels A-E. A comparative analysis with a set concentration of inhibitor (22.4 μg/ml) is shown in panel F for all patients that displayed reactivity towards both TG2 and TG6.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

Neurological disorders have only recently been recognised as potential presenting manifestations of coeliac disease. These include cerebellar ataxia, peripheral neuropathy, chorea, ataxia with myoclonus, myopathy headaches with white matter abnormalities on brain MRI, epilepsy with cerebral calcifications, anxiety/depression and Stiff-person syndrome. Neurological disorders occur with a frequency of up to 10% in coeliac disease patients (Lagerqvist et al., 2001. J. Intern. Med. 250: 241-8). Ataxia and peripheral neuropathy are the most common manifestations with about 35% each (Hadjivassiliou et al., 2002. J. Neurol. Neurosurg. Psychiatry 72: 560-563). Some recent studies suggest that there is no correlation between motor neuron disease and polyneuropathies with either coeliac disease or TG2 IgA (Renzi et al., 2006. Acta. Neurol. Scand. 114: 54-58; Rosenberg and Vermeulen, 2005. J. Neurol. Neurosurg. Psychiatry 76: 1415-1419). Others however have confirmed a higher prevalence of both coeliac disease (at least 8-10%) and the presence of antigliadin antibodies (34%) in patients with otherwise idiopathic axonal neuropathies (Hadjivassiliou et al., 2006. J. Neurol. Neurosurg. Psychiatry 77: 1262-1266; Chin et al., 2003. Neurology 60: 1581-1585). The presence of an enteropathy is not a prerequisite for the diagnosis of gluten sensitivity as neurological disorders are found in asymptomatic patients with gluten sensitivity that show improvement in their condition on a gluten-free diet (Hadjivassiliou et al., 2003. J. Neurol. Neurosurg. Psychiatry 74: 1221-1224). Neurological dysfunction may be the sole presenting feature of gluten sensitivity and only a third of such patients have evidence of an enteropathy on duodenal biopsy. These patients do, however, have circulating anti-gliadin antibodies (IgG and/or IgA) and the majority but not all express the HLA DQ2 or DQ8 (Hadjivassiliou et al., 2003. Brain 126: 685-691).

In common with other manifestations of gluten sensitivity, there is substantial evidence to suggest that the mechanism of neural damage is immune-mediated and, as in coeliac disease, there is strong association with other autoimmune diseases (Sategna Guidetti et al., 2001. Gut 49: 502-505). Both, a humoral as well as cell mediated immune responses are seen in patients with gluten ataxia, a non-genetic sporadic cerebellar ataxia associated with the presence of circulating antigliadin antibodies. The humoral immune response in gluten ataxia comprises of a number of autoantibodies, the majority of which may be represented by the anti-gliadin antibodies possibly reacting with unknown self-antigen(s) (Hadjivassiliou et al., 2002. Neurology 58: 1221-1226). Up to 50% of these patients have oligoclonal immunoglobulin bands in cerebrospinal fluid electrophoresis, evidence of intrathecal antibody production (Hadjivassiliou et al., 2003. Brain 126: 685-691). In addition, these gluten ataxia patients have circulating anti-Purkinje cell antibodies in their sera, although the target antigen is currently unknown. Anti-TG2 IgA deposits have been demonstrated in the small intestine and brain of patients with gluten ataxia but not in control patients with other forms of ataxia (Hadjivassilliou et al., 2006. Neurology 66: 373-377). A role of these antibodies in the pathophysiology of the ataxia is suggested by clinical improvement with immunoglobulin therapy (Sander et al., 2003. Lancet 362: 1548; Burk et al., 2001. Ann. Neurol. 50: 827-828). Evidence for a T-cell response in gluten ataxia patients comes from post mortem studies. Perivascular lymphocytic infiltration of cerebellar tissue by both CD4⁺ and CD8⁺ T-cells was observed in two cases studied at post-mortem (Hadjivassiliou et al., 1998. Lancet 352: 1582-1585). This inflammatory infiltrate predominantly affects the cerebellum with resulting loss of Purkinje cells. Also, levels of the chemokine CXCL10, a T-cell chemoattractant, have been shown to be elevated in the cerebrospinal fluid (CSF) of patients with gluten ataxia compared to controls (Hadjivassiliou at al., 2003. Neurology 60: 1397-1398), supporting a role for a T-cell mediated immune response within the central nervous system.

In coeliac disease, the immune response is primarily driven by gluten-specific CD4⁺ T-cells, which have been isolated from both the small intestine and peripheral blood of patients (Sollid, 2002. Nature Reviews 2: 647-655). However, activation of CD4⁺ T-cells and the presence of antibodies, features characterising the latent stage of the disease, is not sufficient to induce tissue destruction. The effector phase of the disease is mediated by intraepithelial TCRαβ⁺ CM8⁺ T-cells activated by the induction of MHC-like molecules such as MIC on stressed epithelial cells (Sollid and Jabri, 2005. Curr. Opin. Immunol. 17: 595-600). The role of the T-cell mediated immune response in the pathogenesis of neurological dysfunction associated with gluten sensitivity remains to be elucidated.

Neurological dysfunction may be the consequence of development of an immune response primarily targeting an antigen in the central nervous system (CNS). However, an important aspect to consider is the protection of the CNS by the blood brain barrier. Nevertheless, a number of CNS disorders have been shown to be directly linked to autoantibodies (Lang et al., 2003. Curr. Opin. Neurol. 16: 351-357). Reaction of TG2-IgA with TG2 in the blood brain barrier (Aeschlimann and Paulsson, 1991. J. Biol. Chem. 266: 15308-15317) may induce alterations in its integrity and lead to subsequent reaction of autoantibodies with neuronal epitopes. As TG2 is expressed in the brain, and its expression correlated with the neuronal stress response in neurodegenerative diseases (polyglutamine expansion diseases, Alzheimer's, Parkinson's and supranuclear palsy) (Kim et al., 2002. Neurochem. Int. 40: 85-103) it is plausible for TG2 to be this neuronal autoantigen.

However, the inventors have obtained evidence that suggests an alternative explanation. One of the inventors has cloned the transglutaminase isoforms TG5, TG6 and TG7 (WO 02/22830) and as demonstrated in Example 1 of the present application found that one of these, TG6, is predominantly expressed by a subset of neurons in the central nervous system including Purkinje cells. The preferential expression of TG6 in neural tissue provides a clear possibility that this enzyme could be involved in the pathogenesis of autoimmune disorders, and particularly in the pathogenesis of autoimmune disorders with neurological symptoms. As detailed in the examples, the inventors were able to demonstrate a correlation between an immune response to TG6 and brain pathology. Therefore, the fact that only a subset of coeliac disease patients develop neurological dysfunctions is likely to relate to the specificity of the immune response and/or autoantibodies.

In one embodiment, autoantibodies in suitable body fluids are detected by means of an immune reaction with a novel antigen, transglutaminase 6 (TG6). The antigen may adopt the form of native or denatured protein, or fragments or peptides thereof, or may be presented in the form of mixtures or extracts containing TG6 or parts thereof. Most preferably, the Ca²⁺-bound activated form of the enzyme is used. Alternatively an active form of the transglutaminase can be prepared by reaction with a glutamine substrate analogue that forms a stable adduct or by slow hydrolysis to stabilize the active conformation. The method provided may be used for the diagnosis of diseases, or the monitoring and assessment of the effectiveness of therapy, especially autoimmune diseases and in particular autoimmune diseases with neurological symptoms, preferably but not exclusively of the cereal protein sensitive type including ataxia and neuropathy.

As used in this application the term transglutaminase 6 includes human or animal transglutaminase 6 and also includes naturally produced, recombinantly produced, or chemically synthesized transglutaminase 6 or parts thereof. Thus one embodiment is directed to the use of transglutaminase 6, or an antigenically active fragment thereof, for the detection of autoantibodies reacting with transglutaminase 6 for the diagnosis of an autoimmune disorder characterized by the presence of autoantibodies reacting with transglutaminase 6.

The term “antigenically active fragment” is in general defined as a protein fragment that is able to elicit an antibody response and is able to be bound by an antibody. In regard to TG6 an “antigenically active fragment” can be further defined as an arrangement of amino acid residues forming part of the hydrophilic surface of TG6, in particular the surfaces exposed upon enzyme activation, either in the form of a continuous peptide sequence or amino acids held together by an artificial backbone that mimics their arrangement in the native protein. An “antigenically active fragment” can also be a portion of TG6 with gliadin peptides bound to its active site, which constitute common T cell epitopes.

Preferably the autoimmune disorder is a neurological disorder or is characterized by neurological dysfunction. Further preferably the autoimmune disorder is a food protein sensitivity disorder. Further preferably the autoimmune disorder is a cereal protein sensitivity disorder. Further preferably the cereal protein disorder is a gluten sensitivity disorder. Further preferably the autoimmune disorder is a paraneoplastic neurological syndrome.

Preferably the gluten sensitivity disorder is cerebellar ataxia, peripheral neuropathy, myopathy, ataxia with myoclonus, myelopathy, cerebral calcifications, headache with white matter abnormalities, dementia, chorea or Stiff-person syndrome.

Preferably the autoimmune disorder characterised by neurological dysfunction is anxiety, depression, brainstem encephalitis, cerebral vasculitis, chorea, dementia, epilepsy, cerebral calcifications, headache with white matter abnormalities, neuromyotonia, myasthenia gravis, myopathy, peripheral neuropathy, a paraneoplastic syndrome, progressive multifocal leukoencephalopathy, progressive myoclonic encephalopathy, schizophreniform disorder or stiff-person syndrome.

Most preferably the autoimmune disorder characterised by neurological dysfunction is immune mediated ataxia, encephalitis, cerebral vasculitis, neuromyotonia, myasthenia gravis, polymyositis, immune mediated peripheral neuropathy, a paraneoplastic syndrome, or stiff-person syndrome.

Preferably the autoimmune disorder characterised by neurological dysfunction is ataxia, ataxia with myoclonus, encephalitis, polymyositis, epilepsy with occipital cerebral calcifications, peripheral polyneuropathy, loss of sensation or muscle weakness.

Preferably the neurological dysfunction is characterized by ataxia, ataxia with myoclonus, anxiety, depression, dementia, epilepsy with occipital cerebral calcifications, headache with white matter abnormalities, peripheral polyneuropathy, loss of sensation or muscle weakness.

Preferably the paraneoplastic neurological syndrome is paraneoplastic cerebellar degeneration, paraneoplastic encephalomyelitis, paraneoplastic opsoclonus-myoclonus, cancer associated retinopathy, paraneoplastic stiff-person syndrome, paraneoplastic necrotizing myelopathy, a motor neuron syndrome including amyotrophic lateral sclerosis (ALS) and subacute motor neuronopathy, subacute sensory neuronopathy, autonomic neuropathy, acute sensorimotor neuropathy, polyradiculoneuropathy (Guillain-Barré), brachial neuritis, chronic sensorimotor neuropathy, a sensorimotor neuropathy associated with plasma cell dyscrasias, vasculitic neuropathy or neuromyotonia.

Preferably the autoantibodies are detected in a patient body fluid sample. Preferably the patient body fluid sample is a sera sample or a cerebrospinal fluid sample. Further preferably the autoantibodies are detected in a patient tissue sample.

Preferably transglutaminase 6 used to detect the autoantibodies is purified from human tissue. Further preferably the transglutaminase 6 used to detect the autoantibodies is purified from animal tissue. Further preferably the transglutaminase 6 is produced by recombinant DNA technology, based on either the human or an animal or a mixed gene sequence.

Preferably the antigenically active fragment of transglutaminase 6 is a synthetic peptide.

In practice the transglutaminase 6 used to detect autoantibodies can include a mixture of any of a transglutaminase 6 purified from human tissue, a transglutaminase 6 purified from animal tissue, a transglutaminase 6 produced by recombinant DNA technology and one or more synthetic peptides of an antigenically active fragment of transglutaminase 6.

In one embodiment the detection of transglutaminase 6 autoantibodies can be combined with the detection of autoantibodies to one or more further transglutaminase isoforms. The one or more further transglutaminase isoforms can be selected from transglutaminase 1, transglutaminase 2, transglutaminase 3, transglutaminase 4, transglutaminase 5, transglutaminase 7, coagulation factor XIII or erythrocyte membrane protein band 4.2.

Also provided is a method of detecting an autoimmune disorder characterized by the presence of autoantibodies reacting with transglutaminase 6 and the absence of autoantibodies to transglutaminase 2 by the use of transglutaminase 6 and transglutaminase 2, or antigenically active fragments thereof, for the detection of autoantibodies reacting with transglutaminase 6 and transglutaminase 2 for the diagnosis of an autoimmune disorder characterized by the presence of autoantibodies reacting with transglutaminase 6 and the absence of autoantibodies to transglutaminase 2. Preferably the autoimmune disorder is a cereal protein sensitivity disorder and most preferably the cereal protein sensitivity disorder is coeliac disease.

Also provided is a method of detecting an autoimmune disorder characterized by the presence of autoantibodies reacting with transglutaminase 3 and the absence of autoantibodies to transglutaminase 2 by the use of transglutaminase 3 and transglutaminase 2, or antigenically active fragments thereof, for the detection of autoantibodies reacting with transglutaminase 6 and transglutaminase 2 for the diagnosis of an autoimmune disorder characterized by the presence of autoantibodies reacting with transglutaminase 3 and the absence of autoantibodies to transglutaminase 2. Preferably the autoimmune disorder is a cereal protein sensitivity disorder and most preferably the cereal protein sensitivity disorder is coeliac disease.

Any method and device suitable for the diagnostic detection of proteins or antibodies in samples of body fluids or in tissue samples can be used to detect autoantibodies reacting with transglutaminases. Examples of such methods and devices include: EIA/ELISA, LiA, FiA, RIA, IRMA, IEMA/EIA, ILMA, IFMA, immunodiffusion, Western-blot, Dot-blot, immunohistochemistry, protein chips or protein arrays. Thus another aspect is directed to protein chips and protein arrays containing at least transglutaminase 6 preferably in an immobilized form. Such chips and arrays may preferably contain further transglutaminase isoforms selected from transglutaminase 1, transglutaminase 2, transglutaminase 3, transglutaminase 4, transglutaminase 5, transglutaminase 7, coagulation factor XIII or erythrocyte membrane protein band 4.2, preferably in an immobilized form.

Another aspect is directed to a kit for the diagnosis of an autoimmune neurological disorder characterized by the presence of autoantibodies reacting with transglutaminase 6, the kit including transglutaminase 6 or an antigenically active fragment thereof and the use of a kit for the diagnosis of an autoimmune neurological disorder characterized by the presence of autoantibodies reacting with transglutaminase 6, the kit including transglutaminase 6 or an antigenically active fragment thereof.

The kit can also include one or more further transglutaminase isoforms selected from transglutaminase 1, transglutaminase 2, transglutaminase 3, transglutaminase 4, transglutaminase 5, transglutaminase 7, coagulation factor XIII or erythrocyte membrane protein band 4.2.

Preferably the autoimmune neurological disorder is one of the disorders as recited above.

A further aspect is directed to a kit for the diagnosis of a cereal protein sensitivity disorder characterized by the presence of autoantibodies reacting with transglutaminase 6 and the absence of autoantibodies to transglutaminase 2, the kit including transglutaminase 6 and transglutaminase 2, or antigenically active fragments thereof and the use of a kit for the diagnosis of a cereal protein sensitivity disorder characterized by the presence of autoantibodies reacting with transglutaminase 6 and the absence of autoantibodies to transglutaminase 2, the kit including transglutaminase 6 and transglutaminase 2, or antigenically active fragments thereof. Preferably the cereal protein sensitivity disorder is coeliac disease.

A further aspect is directed to a kit for the diagnosis of a cereal protein sensitivity disorder characterized by the presence of autoantibodies reacting with transglutaminase 6 and the absence of autoantibodies to transglutaminase 3, the kit including transglutaminase 6 and transglutaminase 3, or antigenically active fragments thereof and the use of a kit for the diagnosis of a cereal protein sensitivity disorder characterized by the presence of autoantibodies reacting with transglutaminase 6 and the absence of autoantibodies to transglutaminase 3, the kit including transglutaminase 6 and transglutaminase 3, or antigenically active fragments thereof. Preferably the cereal protein sensitivity disorder is coeliac disease.

The above kits can present the transglutaminase or antigenically active fragment thereof in a form suitable for use in, for example, the following methods: EIA/ELISA, LiA, FiA, RIA, IRMA, IEMA/EIA, ILMA, IFMA, immunodiffusion, Western-blot, Dot-blot, immunohistochemistry, protein chips or protein arrays.

### EXAMPLES

Whilst the role of transglutaminases in degenerative neurological diseases has been intensely investigated, their role in the development of immune mediated neurological dysfunction other than the role of TG2 in gluten sensitivity has not been evaluated. Thus in addition to the neurological manifestations of gluten sensitivity we explored transglutaminase-linked autoimmune responses in patients with paraneoplastic neurological syndromes and patients with stiff person syndrome.

### Example 1

### Expression of TG6 Isoform in Neurons of Central Nervous System

Cloning of mouse TG6—Mouse brain (BALB/c strain) was dissected, rinsed in PBS and immediately frozen on dry ice. The frozen brain was homogenised in 1 ml of Tri Reagent (Sigma) using a teflon pestle and total RNA was prepared by chloroform extraction and isopropanol precipitation following the manufacturer's instructions. cDNA was synthesised from 2 μg total RNA by reverse transcription using 200 units of SuperScript II RNase H⁻ Reverse Transcriptase (Invitrogen) and 5 μM oligo(dT)₁₅ primer in a total volume of 20 μl. For the cloning of mouse TG6, a series of gene-specific oligonucleotides modelled from the human TG6 sequence (WO 02/22830) were used to isolate overlapping DNA fragments by PCR. The full-length nucleotide sequence of mouse TG6 was deduced and deposited into the GenBank™/EBI Data Bank under accession number AY159126.

In situ hybridization—A 325 bp fragment corresponding to the 3′ end of mouse TG6 was generated by PCR (nucleotides 1682 to 2007 in GenBank AY159126). The fragment was cloned into the pCRII vector using TA cloning (Invitrogen). For in vitro transcription, the cDNA fragment with flanking RNA polymerase promoters was excised by restriction with PvuII and AflIII and isolated using the QIAquick Gel extraction kit (Qiagen). Digoxigenin-UTP (DIG)-labelled, single-strand antisense and sense RNA probes were prepared using the DIG RNA Labelling Kit (Roche) with 1 μg purified DNA fragment and 40 units of either RNA Polymerase SP6 or T7 in 20 μl transcription buffer supplemented with 0.1 mM ATP, 0.1 mM CTP, 0.1 mM GTP, 0.065 mM UTP, and 0.035 mM DIG-11-UTP following the manufacturer's instructions. DNA template was degraded by incubation with 20 units RNase-free DNase I (Roche) for 15 min at 37° C., the reaction terminated by addition of 0.2M EDTA (pH 8.0) to a final concentration of 0.02M, and after supplementation with 0.4M LiCl, the labelled RNA collected by ethanol precipitation. To determine the yield of labelled RNA, the RNA was compared to a dilution series of a DIG-labelled control RNA by spotting onto a Nytran nylon membrane (Schleicher and Schuell) and visualisation using the DIG Nucleic Acid Detection kit (Roche).

Newborn mouse and mouse embryos at gestation days 11, 13 and 16 were fixed in 4% paraformaldehyde in PBS, pH 7.4, at 4° C. overnight. To improve penetration of fixative into the newborn mouse, an excision was made along the abdomen with a scalpel. The mice were transferred to 0.5% paraformaldehyde in PBS containing 0.42M EDTA for 4 days for demineralisation of skeletal tissues, washed in PBS, then processed through a graded ethanol series and paraffin embedded. Sagittal sections of 5 μm thickness were cut, transferred onto gelatin coated glass slides and dried overnight at 50° C. Following deparaffinisation, tissue sections were washed with DEPC treated H₂O, treated with proteinase K (Sigma; 4 μg/ml in 100 mM Tris-HCl, pH 8, 50 mM EDTA) at 4° C. for 15 min before being acetylated with 0.25% (v/v) acetic anhydride in 0.1M triethanolamine, pH 8.0 for 10 min. Sections were prehybridised for 15 min at 37° C. in 5×SSC, followed by hybridisation overnight at 42° C. with 20 ng/μl of the DIG labelled probe in 30 μl hybridisation solution consisting of 50% formamide, 10% dextran sulphate, 5×SSC and 300 μg/ml herring sperm DNA (Sigma) in DEPC treated H₂O (Frame-Seal incubation chambers [MJ Research, Inc.] were used to eliminate evaporation of reagents during hybridisation). Slides were subsequently washed in 2×SSC for 30 min at room temperature, twice in 2×SSC for 20 min at 37° C., and once in 1×SSC for 20 min at 37° C. Hybridised probe was subsequently visualised using the DIG nucleic acid detection kit (Roche) according to the manufacturer's instructions by incubation with alkaline phosphatase-labelled anti-DIG antibody (diluted 1:500) and subsequent development with NBT/BCIP substrate solution for 12 hours.

Isolation of cortical neurons—Cerebral cortex of newborn Balb C mice was dissected on ice in Hanks balanced salt solution (HBSS) and digested for 20 min at 37° C. in 0.1% trypsin, 0.05% DNAse I (Sigma) in HBSS. The tissue was washed and subsequently triturated in HBSS/DNAse I solution to dissociate cells. For culture of neuronal precursors, cells were washed and subsequently maintained in DMEM/F12 containing 2% B27 supplement (Invitrogen) and 20 ng/ml bFGF, 20 ng/ml EGF, 100 units/ml penicillin G and 100 μg/ml streptomycin. Number of vital cells was determined by trypan blue exclusion and for immunocytochemistry, cells seeded at a density of 1×10⁵ cells/well in 12-well plate on laminin-1 coated cover slips and grown for 5 days (37° C./5% CO₂) to induce differentiation. For FACS analysis, cells were washed in 0.5% BSA in phosphate-buffered saline, pH 7.4 (PBS), filtered through 40 μm Falcon cell strainers (Becton Dickinson) to remove remaining aggregates and fixed for 20 min on ice in 2% paraformaldehyde in PBS.

FACS analysis—Cells were permeabilized in PBS containing 0.5% saponin for 20 min on ice. After three washes in PBS containing 0.1% saponin, non-specific binding was blocked in TBS containing 1% BSA and 3mg/ml rabbit anti-mouse IgG (DAKO) and then cells incubated with monoclonal antibodies against glial fibrillary acidic protein (G-A-5, Sigma,15 μg/ml), β-tubulin isoform III bodies (SDL.3D10, Sigma, 20 μg/ml), oligodendrocytes (RIP, Chemicon,1:1000 diluted) and goat anti TG6 antibodies (20 μg/ml) in TBS/BSA overnight at 4° C. Primary antibody binding was detected by incubation with 13 μg/ml FITC-conjugated rabbit anti mouse (MP Biomedicals) and R-phycoerythrin rabbit anti goat (Sigma) secondary antibodies for 60 min at room temperature. Analysis was performed immediately after labelling using a FACScalibur flow cytometer (Becton Dickinson) equipped with an argon laser emission wavelength of 488 nm. FITC and PE signals were identified using 530 and 585 band pass filters, respectively. The analysis was performed using Cell Quest software (Becton Dickinson). Ten thousand events were acquired for each sample. Background level of fluorescence was determined from controls with non-specific IgG (ChromPure goat/mouse IgG, Jackson ImmunoResearch Labs Inc) replacing the primary antibodies.

Results: We previously cloned human TG6 from a carcinoma cell line (WO 02/22830). To obtain a clearer understanding of TG6 expression on a cellular level, in situ hybridisation was performed on sagittal newborn mouse sections. A 325 bp fragment corresponding to the 3′ end of TG6 was used as a probe as this area has the least homology between the different TGs and a similar human probe gave no cross-hybridisation with other TG gene products in Northern blotting. In situ hybridisation revealed that TG6 is expressed in the brain, within the cell layers containing the neuronal cell bodies of the cerebral cortex (particularly layers II-IV containing granular neurons and pyramidal cells), and the cerebellum (Purkinje cells). TGase 6 was also expressed in neurons of the spinal cord and the retinal cells of the eye.

After identifying that TG6 is expressed predominantly in the central nervous system in the developed organism, we were interested to identify whether the induction of TG6 expression correlated with any specific events in development. While the central nervous system is the first organ system to develop and to differentiate, it is also one of the last to be completed. Simplistically however, the primary parts of the brain can be identified soon after the neural groove, neural plate and head process stage at embryonic day 7.5, and by embryonic day 14 the brain is typically that of a mammal. In situ hybridisation was therefore carried out on mouse embryos at days 11, 13.5 and 16 of gestation (the 11, 13.5 and 16 day mouse embryo is comparable to the 30 day, 38 day, and 10.4-week human embryo, respectively). The walls of the primitive brain divide into an inner ependymal, an intermediate mantel, and outer marginal layer by day 10, whereby the ependymal layer that ultimately forms the lining of the ventricles of the brain is the thickest layer. At embryonic day 11, active proliferation of neuroblastic cells occurs in the walls of the entire central nervous system and these begin to occlude some of the neural cavities. Up to day 11, the major neuroblastic activity is occurring behind the hindbrain where cranial ganglia V to IX develop. Little TG6 expression was detected in the brain at day 11 while extensive labelling was seen in the developing spinal cord. By day 13, TG6 expression was apparent in several parts of the brain and strong staining could be detected in regions undergoing neuronal differentiation such as the mesencephalon. From days 13-16, the major neuroblastic activity occurred in the cerebral cortex (telencephalon) where cells from the mantle layer migrate into the overlying marginal zone to form the neopallial cortex which will become the outer grey matter of the cerebral hemispheres. By day 16, TGase 6 was highly expressed in the cerebral cortex and the expression pattern was comparable to the expression in the fully developed brain. Induction of TG6 expression appears to correlate both spatially and temporally with neurogenesis.

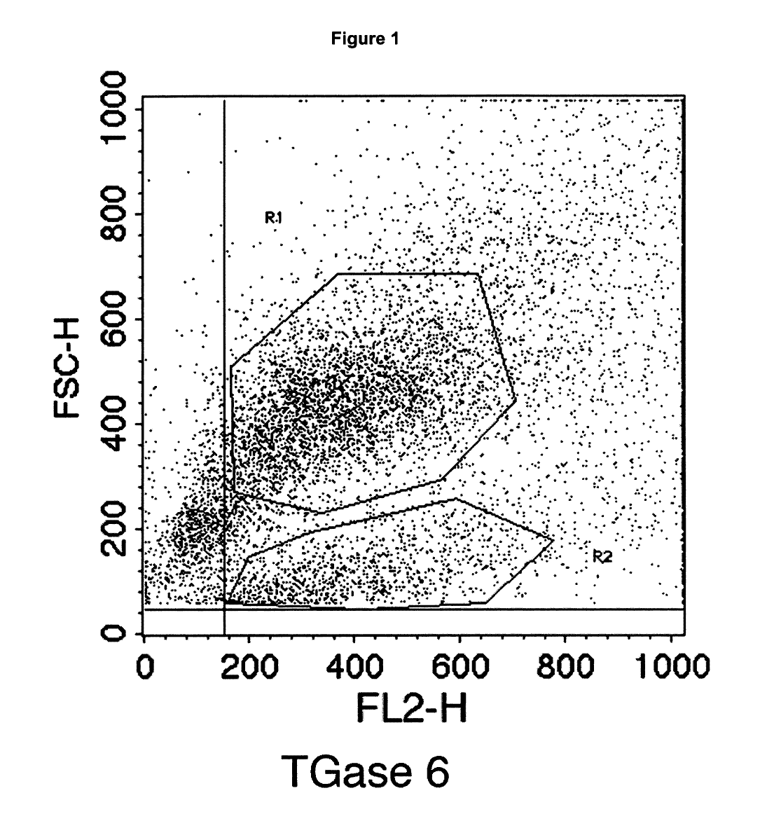

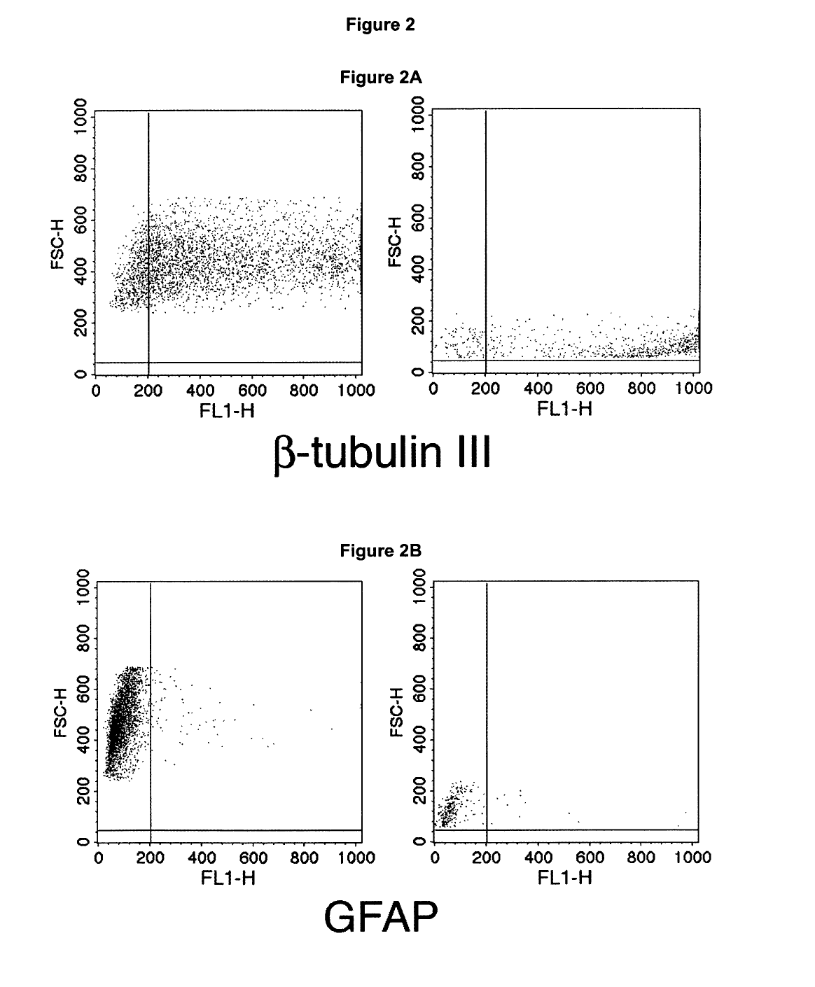

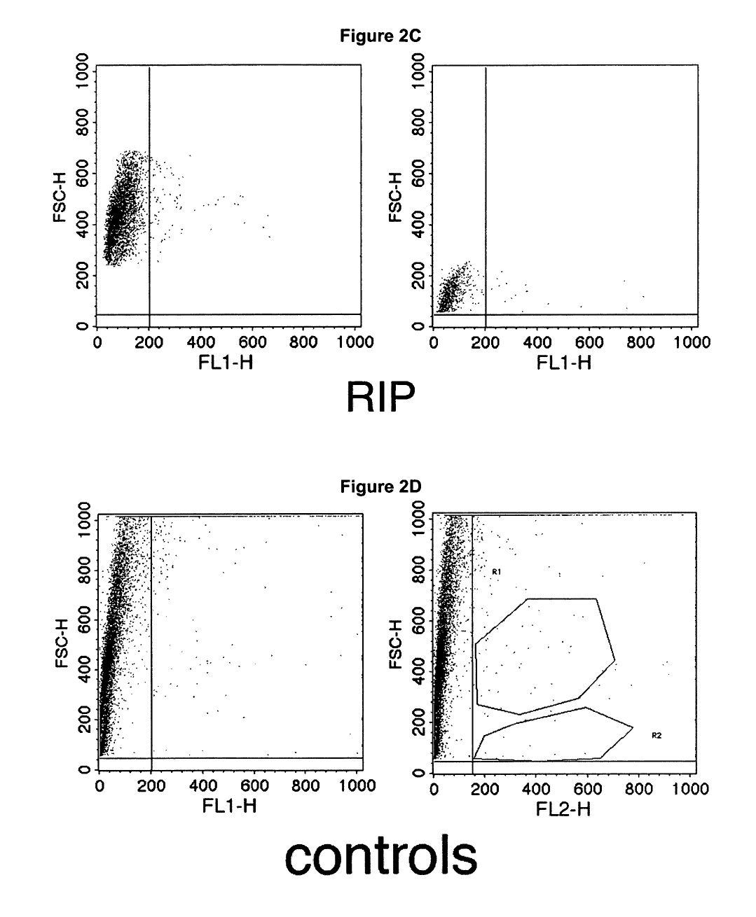

To further characterize the cell population expressing TG6, we used flow cytometry and specific antibodies against intracellular markers to discriminate between different cell types such as neurons, astrocytes or microglial cells. Cells of the neuronal lineage were identified using antibodies against β-tubulin III (Tuj-1), astrocytes with antibodies to glial fibrillary acidic protein (GFAP) and oligodendrocytes with RIP-antibodies, a method verified to be reliable for characterisation of CNS-derived cells (Sergent-Tanguy et al., 2003. J. Neurosci. Meth. 129: 73-79). Polyclonal antibodies to TG6 were raised against a synthetic peptide corresponding to the connecting loop between the catalytic core and β-barrel 1 domain of TG6, purified by affinity chromatography on the synthetic peptide and verified to be specific by immunoblotting of recombinant protein and tissue extracts. Cells were isolated from the cerebral cortex of newborn mice and for analysis using FACS, immediately fixed, permeabilized and double-labelled with antibodies to TG6 and to one of the cell markers. Physical parameters were used to distinguish neurons astrocytes and microglial cells as they differ in size and morphology. Therefore forward scatter (FSC), representing cell size, was plotted as a function of fluorescence intensity for TG6 labelling. Within the broad distribution of cells expressing TG6, two clusters of cells of different size were apparent and were gated (FIG. 1, R1 and R2). Further analysis of gated cells for expression of cell markers showed that both clusters were exclusively positive for β-tubulin III indicating that they are derived from the neuronal lineage and represent different neuronal populations (see FIG. 2). Immunohistochemistry on in vitro differentiated cells confirmed the absence of TG6 from the astroglial and oligodendroglial lineage and expression in a subset of neuronal cells.

### Example 2

### Production of Recombinant Human Transglutaminases

Generation of expression constructs—A full-length cDNA encoding human TG6 was obtained by PCR from poly(A⁺)RNA isolated from the lung carcinoma cell line H69 as previously described (WO 02/22830). Briefly, overlapping PCR fragments were amplified, TA-cloned and the full-length cDNA constructed by subcloning the overlapping fragments into the pCRII vector (Invitrogen) using appropriate restriction endonucleases. Sequence analysis revealed two single-nucleotide deletions (C75 and G1568). The mutations were corrected by site specific insertion mutagenesis using the QuickChange XL Site Directed Mutagenesis kit (Stratagene). Finally, the coding sequence was subcloned into derivatives of the prokaryotic expression vector pRARE (Moralejo et al., 1993. Bacteriol. 175: 5585-5594) for rhamnose-regulated expression in E. coli. cDNAs for TG2 and TG3 were subcloned into the same expression vector. A His6-tag was added to the native sequence for purification of the recombinant proteins by Ni²⁺-chelating affinity chromatography.

Protein expression—E. coli BL21 transformed with the construct containing the full-length human TG2, TG6 or TG3 were inoculated into 50 ml of modified Luria-Bertani (LB) broth (10 g/l tryptone, 5 g/l yeast extract, 5 g/l NaCl, pH 7.2) containing 100 μg/ml ampicillin, and grown overnight at 37° C. in a shaking incubator. The overnight culture was then expanded to 1 litre with LB broth and grown in baffled flasks at 37° C. and 220 rpm to OD₆₀₀ of 0.6 prior to chilling to 20° C. and induction of transgene expression by addition of rhamnose to a final concentration of 0.5% (0.1% for TG6). After incubation for a further 6-24 h (depending on transglutaminase type) at 20° C. the bacteria were collected by centrifugation at 3,000×g for 20 min, resuspended in buffer A, 50 mM Na₂HPO₄, pH 8.0, 300 mM NaCl (or 50 mM MOPS, pH 6.8, 500 mM NaCl, 10 mM glutathione and 30% glycerol for TG6), to obtain a 15% cell suspension, and the expressed protein harvested by lysis of the cells using a ‘French-Press’ (1000 Psi). The lysate was cleared from insoluble material by centrifugation at 11,500×g for 30 min at 4° C. and applied to a 1 ml HisTrap™HP column (Amersham Bioscience) equilibrated in buffer A at 4° C. and a flow rate of 0.5 ml/min. The resin was washed, initially with buffer A until OD₂₈₀ of less than 0.001 was reached and then with 100 ml of 90% buffer A and 10% buffer B (50 mM Na₂HPO₄, pH 8.0, 300 mM NaCl and 300 mM imidazole) (buffer A containing 50 mM imidazole for TG6) before elution of the fusion protein with a mixture of 50% buffer A and 50% buffer B (50 mM MOPS, pH6.8, 300 mM NaCl, 5 mM DTT, 500 mM imidazol, and 10% glycerol for TG6) while collecting 1 ml fractions. Fractions were analysed by SDS PAGE and immunoblotting for His-tagged protein, relevant fractions pooled and dialysed extensively against buffer C (20 mM Tris/HCl, pH 7.2, 1 mM EDTA, 100 mM NaCl) (20 mM Tris/HCl, pH 8.0, 300 mM NaCl, 5 mM DTT, 10% glycerol for TG6). When desired, enzymes were purified further by ion exchange chromatography. Briefly, 5 ml aliquots were applied onto a HR10/10 column packed with Resource Q15 (Amersham Bioscience) for FPLC equilibrated in buffer C. The enzyme was eluted in a single sharp peak with a 20 volume gradient of 100-700 mM NaCl, pooled, dialysed into buffer C, concentrated to ˜2mg/ml using Centriprep-YM30 (Amicon) concentrators and stored at −20° C.

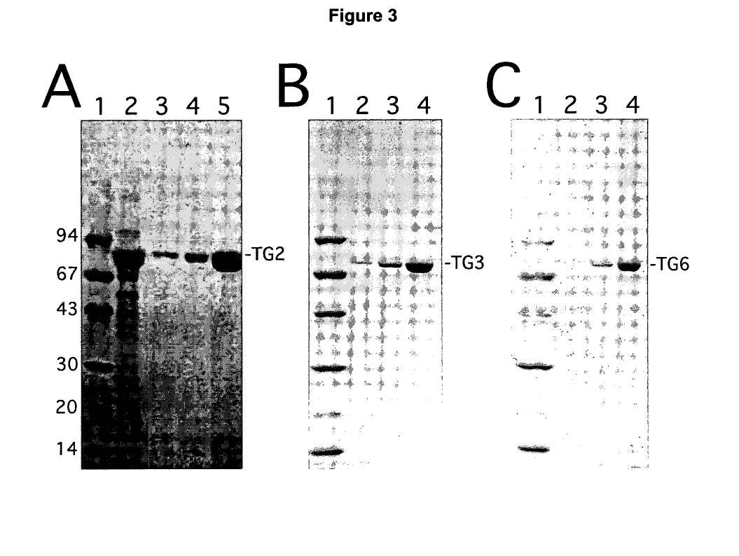

Results: Transglutaminase 2, 3 and 6 were expressed as a fusion protein with N-(TG2, TG3) or C-terminal (TG6) hexahistidine-tag for effective purification. We established a protocol for purification of proteins by sequential Ni-chelating and ion exchange chromatography and were able to produce enzymatically active TG2 and TG3 on the mg and TG6 on the 100 μg scale. The purified proteins gave a single band on Coomassie blue stained SDS-PAGE gels (FIG. 3) and their identity was verified by demonstrating enzymatic activity, immunoreactivity with the respective antibodies, and peptide fingerprinting and/or sequencing using MALDI-TOF mass spectrometry (MS) and tandem MS (more than 50% coverage of the sequence yielding MASCOT scores >800).

### Example 3

### Detection of Antibodies to TG Isoforms in Sera of Control Subjects and Patients with Coeliac Disease or Unexplained Neurological Dysfunctions

Patients—Sera of patient groups with neurological dysfunction: 16 gluten ataxia-without enteropathy (GA); 14 gluten ataxia with enteropathy (GAE); 16 peripheral neuropathy (GN); 3 stiff person syndrome (SMS) and 4 paraneoplastic syndrome (Pneo) were analysed and compared with sera of various control groups: 16 genetic ataxia (GenA); 16 classical coeliac disease (CD) and 18 healthy controls (N). All patients had been examined at the Gastroenterology and Neurology Departments of the Royal Hallamshire Hospital, Sheffield, UK. Collection and analysis of sera has been approved by the research ethics committee (REC 06/Q2307/6).

The gluten ataxia group was defined as sporadic cerebellar ataxia associated with the presence of antigliadin antibodies and the absence of an alternative etiology for ataxia. This group was split into a subgroup with enteropathy (GAE) and a subgroup without enteropathy (GA). These patients are characterised by loss of motor coordination resulting in inability to execute movements with accuracy. This results in unsteadiness on walking, clumsiness in performing limb movements and a tendency to fall. The cerebellum is responsible for coordination and is the organ affected in ataxias. A group of patients with ataxia of known genetic origin served as a control. Peripheral neuropathy is a progressive dysfunction of the nerves that carry information to and from the spinal cord. This produces pain, loss of sensation and muscle weakness. Gluten sensitive peripheral neuropathy (GN) is prevalent among patients with idiopathic peripheral neuropathy at about 34% (Hadjivassiliou et al., 2006. J. Neurol. Neurosurg. Psychiatry 77: 1262-1266).

Stiff person syndrome (SMS) is a rare disorder characterized by severe progressive muscle stiffness of the trunk and lower limbs with painful spasms. An autoimmune aetiology is suggested by the association with HLA DR3, DR4 and DQ2, the presence of serum anti-GAD (glutamic acid decarboxylase) antibodies, and the benefit from intravenous immunoglobulin therapy (Dalakas et al., 2001. New Engl. J. Med. 26: 1870-1876).

The paraneoplastic group (Pneo) included patients with neurological dysfunction as a consequence of various malignancies including lymphoma or small cell carcinoma of lung or ovarian origin. Whilst neuronal antibodies that recognise antigens on transformed cells have been well characterised (anti-Hu, anti-Yo), these antibodies have not been found to be pathogenic. It is possible therefore that neural damage in these syndromes has an alternative explanation involving a transglutaminase. The rationale for this is based on the fact that TG6 has been originally isolated from a cell line derived from a small cell carcinoma of the lung.

Sera of patient groups with neurological symptoms were compared to samples from a group of blood donors of unknown history (N) and a group of diagnosed coeliac disease patients (CD).

Enzyme Linked Immunosorbent Assay (ELISA)—High capacity protein binding 96-well plates (Immulon® 2HB, Thermo Electron) were coated with 100 μl/well of 5 μg/ml antigen (TG2, TG3 or TG6) in TBS (20 mM Tris/HCl, pH 7.4, 150 mM NaCl) overnight at 4° C. All binding steps were followed by 5 rinsing steps with TBS containing 0.01% Tween 20 and all subsequent incubations were carried out at room temperature. Non-specific binding was blocked by incubation with 200 μl/well of 3% BSA in TBS for 60 min. Patient sera were diluted 1:100 in 1% BSA in TBS and any protein aggregates present removed by centrifugation at 10,000×g for 5 min. Coated plates were incubated with 100 μl/well of cleared patient sera for 90 min. After rinsing, serum antibody binding was detected by incubation with 100 μl/well of either peroxidase-conjugated affinity pure goat antihuman IgA (Jackson Immuno Research; diluted 1:2000 in 1% BSA/TBS) or rabbit antihuman IgG (Dako; diluted 1:1000 in 1% BSA/TBS) for 90 min. The reaction was finally developed for 30 min using 5 mM 5-amino-2-hydroxybenzoicacid/NaOH, pH 6.0, 0.005% H₂O₂, as a peroxidase substrate solution (100 μl/well) and stopped by addition of 100 μl 1M NaOH to each well. After 15 min, the absorbance at 490 nm was measured.

All serum samples were analysed twice in triplicates on wells containing antigen or which were blocked with BSA only. A selected negative and a selected positive reference serum, as well as a buffer blank, were also run in parallel on each plate. The BSA only background was subtracted, and the antibody reading expressed in arbitrary units as a percentage of the reference sera.

Inhibition ELISAs followed the protocol above but each serum was titrated to identify the dilution that yielded half maximal binding. The respective sera dilutions were incubated with a concentration series of TG as indicated overnight at 4° C. while shaking and the mixture subsequently added to TG coated plates for 40 min, and the reaction developed as above. Experimental data points are shown in combination with theoretical inhibition curves calculated according to Engel and Schalch, 1980. Mol Immunol 17:675-680.:

$$antigen bound = \frac{\begin{matrix}1 + K ( {c}_{I} + {c}_{Ag} + {c}_{Ab} ) - \\ \sqrt{{( 1 + K ( {c}_{I} + {c}_{Ag} + {c}_{Ab} ) )}^{2} - 4 {K}^{2} {c}_{Ab} ( {c}_{I} + {c}_{Ag} )}\end{matrix}}{2 K ( {c}_{I} + {c}_{Ag} )} ( 1 - {B}_{m i n} ) + {B}_{m i n}$$

whereby concentrations of competitor, coated antigen and antibody are denominated C_{I}, C_{Ag}, and C_{Ab}, respectively. Bₘᵢₙ reflects the level of binding at saturating concentrations of competitor.

Statistics—Based on previous work on coeliac patient sera we know that data from such assays do not show a Gaussian distribution but that the difference between means of positive and negative serum tests is very large. Conservatively assuming that a difference of 50 units will be sufficient to differentiate between the respective groups non-parametric analysis (Mann-Whitney test) indicates that a sample size of 10 per group is sufficient to provide a power of 90% at a significance level of 0.05.

For comparison between patient groups, Kruskal-Wallis nonparametric analysis was used and significance between individual patient groups and healthy controls determined from Dunn's post test. For comparison of inhibition with TG2 or TG6 in ELISAs, Wilcoxon's two-tailed signed ranks test for pairs was used.

Results: Human TG6 (SEQ. ID No. 1) and other transglutaminase isoforms were expressed using recombinant DNA technology and ELISAs performed based on the respective purified proteins for detection of IgG and IgA in serum and cerebrospinal fluid (CSF). Recombinant human proteins were chosen over proteins purified from animal tissue as this has been reported to increase the sensitivity of the assay in the case of TG2 (WO 01/0133). Furthermore, expression in a non-mammalian host cell has been shown to reduce the risk for crossreactivity of sera—and particularly sera from patients with autoimmune disease—with minor impurities in the protein preparation (Sardy et al., 2007. Clin. Chim. Acta 376: 126-135). ELISAs were performed using the same antigen concentration for coating, serum dilution, and reaction time for assay development. At serum dilutions of 1:100 or less, some negative sera showed increased signal while the signal in some positive sera reached a plateau. For sera from gluten ataxia patients, the signal to background ratio was highest at a dilution of 1:100 and hence, this dilution was used for all assays. A small number of sera produced abnormally high signal on wells incubated with blocking agent but in the absence of antigen. High serum immunoglobulin concentrations have been reported to cause false-positive classification in TG2 IgA assay (Villalta et al., 2005. Clin. Chim. Acta 365: 102-109), therefore all assays were carried out in the presence and absence of antigen and the relative signal was used for analysis. A selected positive and negative reference serum was included in each assay to control for assay performance and for data normalization into arbitrary units as a function of the reference samples. In house assay performance was further evaluated against a commercially available clinical assay for TG2 IgA (Genesis Diagnostics) and the mean interassay variation was found to be 11.0% and 3.6% for CD and healthy groups, respectively. We also investigated whether the results were dependent on the conformational state of the antigen. For this purpose, antigen (TG2 or TG6) was pretreated and coated at 4° C. in buffer containing 5 mM CaCl_{2 (Ca}₂-activated form), 1 mM EDTA (reversibly inactivated conformation) or 2M urea (partially denatured form; note, conformational changes induced by urea >1M are irreversible). For TG2 IgA, conformational changes induced by EDTA or urea reduced the signal in positive serum samples from 0.97±0.05 OD to 0.80±0.06 and 0.80±0.06 OD, respectively, and in negative serum samples from 0.12±0.01 OD to 0.10±0.01 and 0.08±0.01 OD, respectively. The reduction in signal was identical with either treatment and ranged from 11 to 30% for individual patients. Similar results were obtained for TG6 IgA and IgG sera analysis. These data are consistent with data in the literature (WO 01/01133) and suggest that antigen conformation has no principal effect on the performance of the test although the signal was significantly higher with the Ca₂-activated form of the enzyme for all patients tested. Samples were strictly kept at 4° C. for coating and it is therefore unlikely that autocatalytic crosslinking played a major role in the enhanced antibody binding. This result is consistent with the recently suggested large conformational change upon enzyme activation (Pinkas et al., 2007. PLoS Biol 5: Dec epub e327) leading to the exposure of masked epitopes which appear to be specifically recognized by coeliac patient antibodies. Also, we carried out parallel ELISAs using TG6 expressed in insect-derived SF9 cells and obtained results comparable to those with E. coli protein preparations providing further evidence that potential crossreactivities with impurities in the protein preparations are insignificant if present.

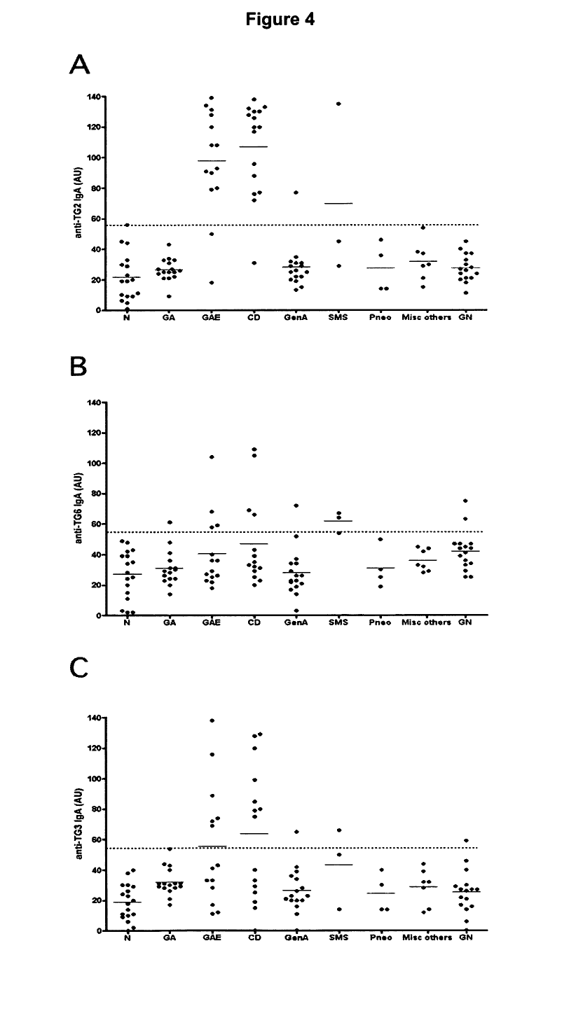

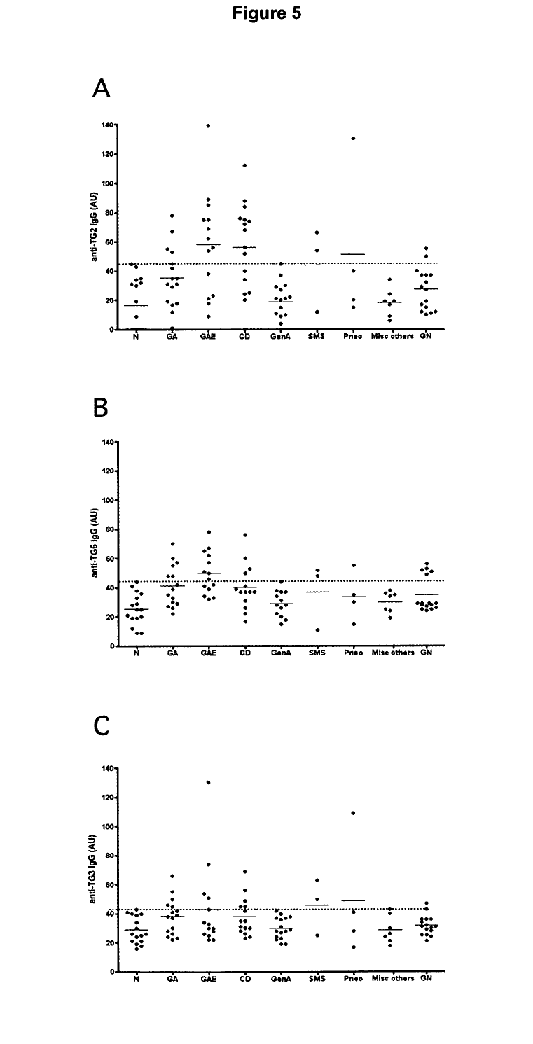

The antibody concentrations (in AU) determined by the ELISAs for TG2, TG6, and TG3 are presented in FIGS. 4 and 5.

The coincidence of the TG2 IgA assay with the clinical diagnosis of coeliac disease was 15/16 (94%) with the remaining individual having antibodies to TG3 only (FIG. 4). 7 (47%) of these 15 patients were also positive for TG3 IgA and 5/15 (33%) had IgA to TG6. IgG titres were generally lower and less predictive with only 10/16 (63%) having a positive result for TG2 IgG (FIG. 5).

Ataxia of the gluten sensitivity type was characterized by an antibody response to gliadin (Table 1). Gluten ataxia patients were further grouped into two subgroups, those with enteropathy and those without, after it became clear that enteropathy correlated with TG2 IgA (FIG. 4). For those with gastrointestinal disease, transglutaminase antibodies were an excellent predictor with 12/14 (86%) having TG2 IgA titres similar to coeliac disease patients and a positive result in the endomysial antibody (EMA) test (Table 1). 50% of these individuals were also positive for TG3 IgA and 33% were positive for TG6 IgA, a pattern identical to that of patients with intestinal manifestation of coeliac disease. This is also consistent with all but 1 (DQ4) of these carrying HLA DQ2 (Table 1). HLA DQ4 is an unusual case which contains only part of the DQ2 complex but is nevertheless consistent with specific recognition of deamidated epitopes. The remaining two individuals of this group (GAE19 & GAE32) were EMA negative and transglutaminase IgA negative but had IgGs to TG6 and TG2 (Table 1) suggesting that based on gastrointestinal symptoms, these individuals may have been assigned to the incorrect group. For the gluten ataxia group without enteropathy, none of the 16 patients tested positive for EMA or TG2 IgA (Table 1). However, 8/18 (44%) (after reassignment of the above 2 individuals) patients had anti-TG6 IgGs and 12/18 (67%) had anti-transglutaminase IgG of one or more type (FIG. 5). It is interesting to note that anti-TG6 IgG was also more prevalent in the ataxia/enteropathy group (50%) than in the coeliac disease group (29%).

The EMA result, the class II HLA type and presence or absence and type of gliadin antibody for the gluten ataxia patients, with and without enteropathy, and for the genetic ataxia patients are summarized in Table 1 below. Also presented in Table 1 is the TG antibody concentration raw data determined by ELISA and used to generate FIGS. 4 and 5. A reading of >=55 AU and of >=45 AU was taken as a positive test result for IgA and IgG, respectively.

TABLE 1

| | | | | | | | | | |
|---|---|---|---|---|---|---|---|---|---|
| | | | | α | α | α | α | α | α |
| | HLA | | α | TG2 | TG6 | TG3 | TG2 | TG6 | TG3 |
| Patient | type | EMA | gliadin | IgA | IgA | IgA | IgG | IgG | IgG |

Gluten ataxia

| | | | | | | | | | |
|---|---|---|---|---|---|---|---|---|---|
| GA 2 | DQ1 | negative | IgG | 27 ± 2 | 30 ± 2 | 31 ± 2 | 42 ± 2 | 26 ± 1 | 39 ± 4 |
| GA 3 | DQ1 | negative | IgA | 33 ± 3 | 48 ± 1 | 44 ± 6 | 55 ± 6 | 60 ± 3 | 45 ± 5 |
| GA 4 | DQ1 | negative | IgG | 24 ± 2 | 28 ± 2 | 29 ± 1 | 53 ± 3 | 42 ± 4 | 46 ± 3 |
| GA 7 | DQ2 | unknown | IgG | 21 ± 3 | 24 ± 2 | 31 ± 2 | 29 ± 5 | 57 ± 1 | 38 ± 3 |
| GA 12 | DQ1 | negative | IgG | 43 ± 3 | 26 ± 1 | 21 ± 1 | 31 ± 5 | 22 ± 1 | 42 ± 2 |
| GA 20 | DQ8 | negative | IgG/IgA | 25 ± 3 | 23 ± 2 | 28 ± 1 | 12 ± 7 | 48 ± 2 | 41 ± 1 |
| GA 21 | DQ1 | negative | IgG/IgA | 9 ± 4 | 24 ± 1 | 29 ± 3 | 0 ± 10 | 27 ± 1 | 24 ± 1 |
| GA 22 | DQ2 | negative | IgG/IgA | 25 ± 1 | 31 ± 4 | 28 ± 2 | 78 ± 4 | 70 ± 1 | 66 ± 5 |
| GA 23 | DQ2 | negative | IgG | 31 ± 2 | 74 ± 3 | 40 ± 4 | 67 ± 4 | 26 ± 1 | 30 ± 2 |
| GA 24 | DQ2 | negative | IgG | 22 ± 1 | 14 ± 2 | 17 ± 2 | 31 ± 2 | 39 ± 2 | 28 ± 1 |
| GA 25 | DQ2 | negative | IgG/IgA | 34 ± 3 | 41 ± 3 | 31 ± 1 | 35 ± 1 | 30 ± 2 | 55 ± 2 |
| GA 26 | DQ2 | negative | IgG/IgA | 25 ± 2 | 36 ± 2 | 26 ± 2 | 18 ± 1 | 48 ± 1 | 22 ± 1 |
| GA 27 | DQ2 | negative | IgA | 26 ± 1 | 31 ± 0 | 30 ± 4 | 45 ± 1 | 35 ± 1 | 26 ± 2 |
| GA 28 | — | negative | IgG | 27 ± 1 | 29 ± 2 | 43 ± 2 | 35 ± 2 | 33 ± 1 | 23 ± 1 |
| GA 29 | DQ2 | negative | IgG | 21 ± 1 | 20 ± 2 | 31 ± 5 | 19 ± 2 | 29 ± 1 | 50 ± 3 |
| GA 30 | DQ1 | negative | IgG | 33 ± 2 | 61 ± 2 | 54 ± 4 | 27 ± 1 | 55 ± 2 | 37 ± 1 |

Gluten ataxia with enteropathy

| | | | | | | | | | |
|---|---|---|---|---|---|---|---|---|---|
| GAE 1 | DQ2 | positive | IgG/IgA | 91 ± 4 | 25 ± 1 | 89 ± 4 | 62 ± 5 | 57 ± 1 | 130 ± 0 |
| GAE 5 | DQ2 | positive | IgG/IgA | 108 ± 4 | 27 ± 1 | 33 ± 4 | 75 ± 2 | 62 ± 2 | 51 ± 1 |
| GAE 8 | DQ2 | positive | IgG/IgA | 79 ± 3 | 22 ± 2 | 11 ± 3 | 56 ± 2 | 32 ± 1 | 25 ± 1 |
| GAE 11 | DQ2 | positive | IgG/IgA | 108 ± 3 | 40 ± 1 | 72 ± 5 | 69 ± 3 | 50 ± 2 | 54 ± 4 |
| | DQ8 | | | | | | | | |
| GAE 14 | DQ2 | positive | IgG/IgA | 120 ± 3 | 36 ± 2 | 17 ± 1 | 38 ± 3 | 46 ± 1 | 28 ± 1 |
| GAE 16 | DQ4 | negative | IgA | 93 ± 4 | 104 ± 5 | 138 ± 15 | 9 ± 8 | 42 ± 4 | 22 ± 1 |
| GAE 17 | — | positive | IgG | 80 ± 3 | 23 ± 1 | 43 ± 5 | 23 ± 6 | 41 ± 1 | 33 ± 1 |
| GAE 19 | DQ2 | negative | IgG | 18 ± 3 | 29 ± 2 | 12 ± 4 | 75 ± 3 | 78 ± 1 | 43 ± 3 |
| GAE 31 | DQ2 | positive | IgG/IgA | 142 ± 8 | 53 ± 1 | 41 ± 4 | 18 ± 3 | 51 ± 3 | 26 ± 1 |
| GAE 32 | DQ2 | negative | IgG | 50 ± 2 | 26 ± 1 | 28 ± 5 | 54 ± 3 | 67 ± 1 | 30 ± 1 |
| GAE 33 | DQ2 | positive | IgG | 90 ± 5 | 18 ± 2 | 33 ± 2 | 85 ± 2 | 34 ± 2 | 22 ± 1 |
| GAE 34 | DQ2 | positive | IgG/IgA | 131 ± 7 | 68 ± 0 | 69 ± 10 | 21 ± 1 | 39 ± 1 | 30 ± 2 |
| GAE 35 | DQ2 | positive | IgG/IgA | 134 ± 7 | 58 ± 1 | 116 ± 1 | 89 ± 4 | 33 ± 1 | 74 ± 2 |
| GAE 36 | DQ2 | positive | IgG | 128 ± 4 | 36 ± 1 | 74 ± 1 | 212 ± 5 | 65 ± 1 | 34 ± 4 |

Genetic ataxia

| | | | | | | | | | |
|---|---|---|---|---|---|---|---|---|---|
| GenA 1 | DQ3 | negative | negative | 22 ± 3 | 23 ± 1 | 28 ± 4 | 11 ± 9 | 37 ± 1 | 23 ± 2 |
| GenA 2 | — | negative | negative | 29 ± 1 | 34 ± 1 | 23 ± 3 | 30 ± 6 | 44 ± 1 | 37 ± 2 |
| GenA 3 | DQ2 | negative | negative | 15 ± 3 | 14 ± 2 | 11 ± 4 | 0 ± 8 | 15 ± 1 | 19 ± 2 |
| GenA 4 | DQ1 | negative | negative | 19 ± 1 | 17 ± 1 | 20 ± 3 | 20 ± 7 | 31 ± 1 | 28 ± 2 |
| GenA 5 | DQ1 | negative | negative | 32 ± 2 | 37 ± 3 | 36 ± 2 | 37 ± 9 | 37 ± 1 | 28 ± 1 |
| GenA 6 | — | negative | negative | 31 ± 1 | 52 ± 0 | 42 ± 3 | 45 ± 3 | 43 ± 2 | 38 ± 3 |
| GenA 7 | DQ1 | negative | negative | 25 ± 4 | 34 ± 1 | 39 ± 3 | 0 ± 9 | 22 ± 1 | 24 ± 2 |
| GenA 8 | DQ2 | negative | negative | 20 ± 3 | 21 ± 2 | 16 ± 4 | 9 ± 6 | 41 ± 4 | 37 ± 2 |
| GenA 9 | DQ2 | positive | IgG/IgA | 77 ± 2 | 72 ± 1 | 65 ± 3 | 22 ± 7 | 28 ± 2 | 42 ± 3 |
| | | on re- | on re- | | | | | | |
| | | examination | examination | | | | | | |
| GenA 10 | DQ1 | negative | negative | 13 ± 2 | 3 ± 3 | 0 ± 3 | 15 ± 6 | 28 ± 2 | 31 ± 2 |
| GenA 11 | DQ1 | negative | negative | 26 ± 4 | 23 ± 2 | 21 ± 3 | 29 ± 6 | 34 ± 1 | 40 ± 2 |
| GenA 12 | DQ2 | negative | negative | 25 ± 3 | 22 ± 2 | 20 ± 2 | 21 ± 7 | 38 ± 1 | 29 ± 1 |
| GenA 13 | DQ2 | negative | negative | 29 ± 2 | 19 ± 2 | 26 ± 4 | 10 ± 3 | 18 ± 1 | 19 ± 2 |
| GenA 14 | DQ8 | negative | negative | 35 ± 1 | 26 ± 1 | 20 ± 3 | 4 ± 4 | 20 ± 1 | 22 ± 2 |
| GenA 15 | DQ2 | negative | negative | 22 ± 1 | 26 ± 2 | 23 ± 1 | 27 ± 4 | 26 ± 1 | 27 ± 2 |
| GenA 16 | DQ3 | negative | b IgA | 31 ± 1 | 29 ± 2 | 34 ± 7 | 21 ± 2 | 43 ± 2 | 36 ± 2 |

For two patients of the gluten ataxia without enteropathy group that were found not to have any anti-transglutaminase antibodies, GA12 and GA21, the diagnosis has in recent clinical follow-up examinations been revised: GA12 has lobar dementia (speech and language) and not gluten ataxia. GA21 was found to have B12 deficiency (which could be related to gluten sensitivity) but the neurological symptoms of ataxia have resolved following B12 injections and hence may have been B12 deficiency related.

Antibodies to TG6 were also prevalent in patients with the rare neurological condition stiff person syndrome, with 3/3 having anti-TG6 IgA and 2/3 anti-TG6 IgG. While one of these had coeliac disease (EMA positive and high TG2 IgA titre), the other 2 had a prevalent IgG response.

In the control groups (16 blood donors and 16 genetic ataxia) only 1 patient had any antibodies to transglutaminase isoforms (GenA 9). This patient had the risk HLA DQ2, moderate titres of IgA to TG2, TG3 and TG6 and is likely to be a clinically silent coeliac disease patient. The patient has subsequent to these findings been re-tested and now shown to be positive for antigliadin antibodies and confirmed to have coeliac disease by intestinal biopsy. Overall, anti-TG6 IgG was more frequent than any other anti-transglutaminase antibodies in patients with neurological dysfunctions.

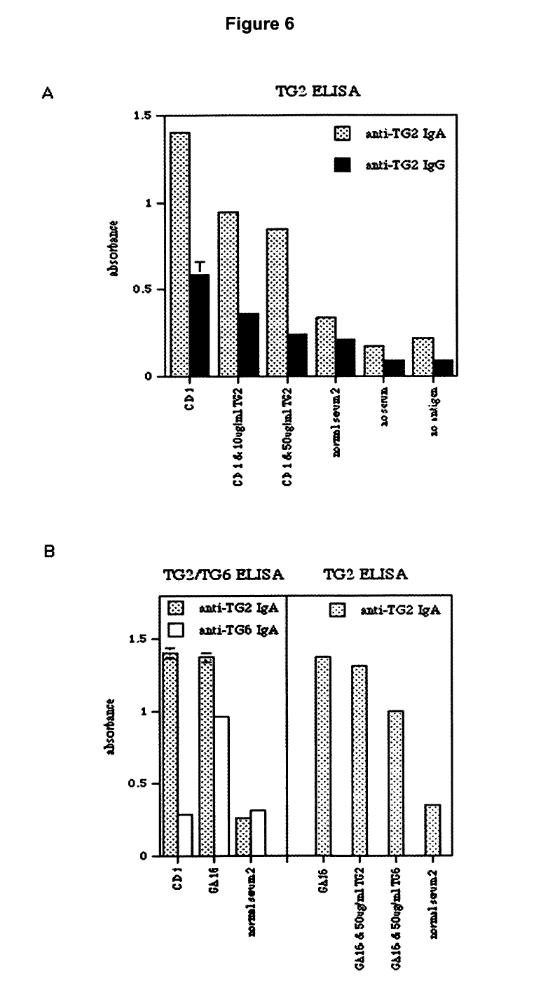

To assess whether isoform-specific antibodies were present or the same antibodies cross-reacted between enzyme isoforms, inhibition studies were carried out on selected sera. Using coeliac disease sera with reaction to TG2 only (FIG. 6B, left panel), we could show a dose-response whereby the highest concentration employed, 50 μg/ml of TG2, completely (93%) blocked IgG detection and partially (52%) blocked IgA detection (FIG. 6A). On the other hand, TG6 was much more effective (37%) than TG2 (6%) in blocking the signal produced by gluten ataxia sera which tested positive for IgA to TG6 and other transglutaminase isoforms (FIG. 6B). This result together with the finding that a number of patients exclusively tested positive for anti-TG6 IgG (Table 1) provides evidence that some patients develop populations of antibodies which are specific for or have greater avidity for TG6 than other enzyme isoforms as has been observed for IgA to TG3 in dermatitis herpetiformis patients (WO 01/01133). Our results demonstrate that a subgroup of patients with neurological dysfunction due to autoimmune processes develop a TG6-specific B-cell response.

### Example 4

### Subsequent Data for the Detection of Antibodies to TG Isoforms in Sera of Control Subjects and Patients with Coeliac Disease or Unexplained Neurological Dysfunctions

Using the same methods as described for Example 3 further data was collected. Patient groups included in the analysis were as follows: Sera from 20 patients with newly diagnosed CD collected before the commencement of a gluten-free diet. CD was confirmed on duodenal biopsy and patients had no evidence of neurological manifestations. Groups with neurological disease included baseline sera from 34 patients with gluten ataxia (defined as otherwise sporadic idiopathic ataxia with positive anti-gliadin antibodies IgG and/or IgA), 15 of these patients had gluten ataxia with enteropathy (GAE) and 19 had gluten ataxia without enteropathy (GA), and also 17 sera from patients with peripheral idiopathic neuropathy positive for anti-gliadin antibodies. A genetic ataxia group served as ataxia disease control. This included 18 patients with either genetically characterised ataxia or clear evidence of autosomal dominant family history of ataxia. A further control group (Misc) included a total of 14 patients with immune-mediated but gluten unrelated disease (vasculitis, viral cerebellitis, paraneoplastic ataxia, GAD ataxia). Finally samples from 19 healthy individuals were used as controls.

Within the group of patients with classical CD, 18/20 had positive serology for anti-TG2 IgA with the remaining two patients having either only IgA type antibodies to TG3 or TG6, respectively. IgG titres, by comparison to IgA were generally lower and a positive test less frequent and always associated with anti-TG2 IgA (Tables 2 and 3). 55% of CD patients tested positive for multiple TG isoforms, 45% for TG3 and 45% for TG6 whereby 35% had antibodies reacting with all 3 isoforms. While the mean antibody concentrations against TG2 were significantly higher than those to other isoforms when comparing groups (Table 2), mean titres were similar when only comparing individuals that tested positive. These results confirm that the B-cell response in gluten sensitivity can be directed to TG isoforms other than TG2 and suggests that frequently antibodies reacting with TG3 or TG6 are present.

Gluten ataxia patients were grouped into two subgroups, those with enteropathy (GAE) and those without (GA). IgA to TG2 was an excellent predictor of the presence of enteropathy with 12/15 GAE patients being positive as opposed to only 1/19 GA patients testing marginally positive. Similarly, while anti-TG3 IgA could be detected in the GAE group with a frequency similar to that in CD, GA patients were not different from controls. This is also consistent with the finding that 79% of GAE patients were EMA positive while all of the GA patients were EMA negative (Table 4). In contrast, the frequency of positive results in the TG6 IgA ELISA was similar for the two groups, i.e. 6/15 (40%) with GAE and 9/19 (47%) with GA. Furthermore, whilst anti-TG6 IgG was only seen in 3/20 CD patients, the prevalence was significantly higher in GA patients, 6/19 (32%), and even higher in GAE patients, 8/15 (53%) (Table 3). Also, some patients tested positive exclusively for IgG class antibodies. The overall prevalence of anti-TG6 IgA and/or IgG was 62% in GA compared to 45% in CD. None of the patients with genetic ataxias or healthy controls had elevated anti-TG antibodies while 1/14 patients with gluten unrelated immune-mediated disease was found to have IgG but not IgA class anti-TG antibodies. The median antibody concentrations were significantly different in patients with gluten sensitivity (CD, GA) as compared to controls (p<0.0005 for IgA, p<0.001 for IgG) whereas no significant differences were seen between the control groups (Table 2). Patients with peripheral neuropathy were not different from controls in all anti-TG IgG assays as well as IgA assays to TG2 and TG3 but had marginally elevated readings in the anti-TG6 IgA ELISA. The significance of this is unclear.

TABLE 2
Concentrations of IgG and IgA (in AU) against TG2, TG3, and TG6 in serum of
healthy controls (HC) and in patients with coeliac disease (CD), gluten ataxia
with enteropathy (GAE), gluten ataxia without enteropathy (GA ), genetic ataxias
(GenA), peripheral idiopathic neuropathy (PN) and various gluten unrelated
autoimmune conditions (Misc). Data is shown as the median, 95% CI of the mean
and significance from Kruskal-Wallis post test analysis.

| | TG2 | | TG3 | | TG6 | |
|---|---|---|---|---|---|---|
| | IgA | IgG | IgA | IgG | IgA | IgG |
| HC | 20.0; | 9.0; | 23.0; | 26.0; | 24.0; | 27.0; |
| (19) | [15.9, | [1.9, | [16.1, | [23.9, | [18.5, | [22.1, |
| | 30.3] | 22.0] | 29.5] | 33.1] | 30.5] | 33.0] |
| CD | 120.0; | 62.0; | 46.0; | 34.0; | 47.0; | 37.0; |
| (20) | [92.7, 122] | [45.6, | [38.0, | [30.7, | [38.2, | [31.4, |
| | p < 0.001 | 82.9] | 76.3] | 45.4] | 67.6] | 45.7] |
| | | p < 0.001 | p < 0.05 | ns | p < 0.05 | ns |
| GAE | 93.0; | 54.0; | 46.0; | 34.0; | 41.0; | 51.0; |
| (15) | [72.6, 113] | [34.3, | [38.3, | [31.9, | [36.0, | [42.0, |
| | p < 0.001 | 73.9] | 78.6] | 62.7] | 73.9] | 57.7] |
| | | p < 0.01 | p < 0.01 | ns | p < 0.05 | p < 0.001 |
| GA | 27.0; | 41.0; | 31.0; | 42.0; | 53.0; | 43.0; |
| (19) | [26.2, | [33.8, | [29.5, | [37.3, | [37.9, | [36.8, |
| | 35.2] | 57.9] | 40.1] | 53.5] | 74.1] | 52.6] |
| | ns | p < 0.01 | ns | p < 0.01 | p < 0.05 | p < 0.01 |
| GenA | 25.5; | 21.0; | 23.0; | 28.0; | 23.0; | 31.0, |
| (18) | [22.2, | [12.6, | [21.6, | [23.6, | [17.0, | [25.9, |
| | 28.3] | 25.4] | 30.3] | 31.9] | 31.7] | 35.9] |
| | ns | ns | ns | ns | ns | ns |
| PN | 26.0; | 29.0; | 27.0; | 31.0; | 39.0, | 29.0, |
| (17) | [22.4, | [20.7, | [19.0, | [28.0, | [33.5, | [28.8, |
| | 31.7] | 35.2] | 34.4] | 35.1] | 50.9] | 39.2] |
| | ns | ns | ns | ns | ns | ns |
| Misc | 29.0; | 19.5; | 30.5; | 29.0; | 36.5, | 34.0; |
| (14) | [22.2, | [12.6, | [21.5, | [19.7, | [28.0, | [26.1, |
| | 36.2] | 48.2] | 34.3] | 48.6] | 48.5] | 41.3] |
| | ns | ns | ns | ns | ns | ns |

TABLE 3
Prevalence of gluten sensitivity detected with IgG
and IgA antibody assays against TG2, TG3 and TG6.

| | TG2 | | TG3 | | TG6 | | |
|---|---|---|---|---|---|---|---|
| | IgA | IgG | IgA | IgG | IgA | IgG | IgG & IgA |
| CD | 90% | 60% | 45% | 20% | 45% | 15% | 45% |
| (20) | | | | | | | |
| GAE | 80% | 53% | 40% | 40% | 40% | 53% | 67% |
| (15) | | | | | | | |
| GA | 5% | 37% | 5% | 32% | 47% | 32% | 58% |
| (19) | | | | | | | |
| GenA | 0% | 0% | 0% | 0% | 0% | 0% | |
| (18) | | | | | | | |
| PN | 0% | 6% | 6% | 0% | 18% | 12% | |
| (19) | | | | | | | |
| Misc | 0% | 7% | 0% | 7% | 7% | 7% | |
| (14) | | | | | | | |
| HC | 0% | 0% | 0% | 0% | 0% | 0% | |
| (19) | | | | | | | |

TABLE 4
Correlation between anti-TG antibodies and endomysial
antibody (EMA) reactivity in GA patients.

| | | | | EMA negative | | |
|---|---|---|---|---|---|---|
| | | | EMA | | | TG2 & |
| | EMA^{a} | | positive | | TG2 & | TG6 |
| | posi- | nega- | TG2 | TG2 | TG6 | IgA or |
| | tive | tive | IgA | IgA | IgA | IgG |
| GAE | 11/15 | 3/15 | 11/11 | 1/3 | 1/3 | 3/3 |
| | (73%) | (20%) | (100%) | (33%) | (33%) | (100%) |
| GA | 0/19 | 17/19 | — | 0/17 | 8/17 | 11/17 |
| | (0%) | (90%) | | (0%) | (47%) | (65%) |

^{a}not known for 1 GAE and 2 GA patients

In this study, we identify a novel TG as the prevalent autoantigen in the CNS in GA and show that among sporadic idiopathic ataxia patients with anti-gliadin antibodies all of those which present with enteropathy and 68% of those without gastroenterological symptoms had circulating anti-TG antibodies while such antibodies were absent in healthy controls or patients with inherited ataxias. Interestingly, the prevalence of the risk HLAs DQ2 and DQ8 differed accordingly in the different ataxia groups examined, with 93% and 72% in GAE and GA, respectively, as opposed to 44% in the genetic ataxia group, with the latter being comparable to the regional population average of 38%. This provides strong evidence for a link between gluten sensitivity and idiopathic ataxia in the vast majority of patients within a group that can be expected to be heterogeneous as classification was solely based on classical anti-gliadin antibody test. In a group of peripheral neuropathy patients with anti-gliadin antibodies no similar correlation could be established although a few patients tested marginally positive for anti-TG antibodies when compared to the other control groups (Table 2).

Autoantibodies against TG2 are responsible for the endomysial (EMA), reticulin (ARA), and jejunal (JEA) reactivity of serum samples from CD patients. Seronegativity of GA patients in these conventional tests appears to reflect the preferential development of Igs specific for TG6 and absence of anti-TG2 IgA, in particular (Table 4). A humoral response to a different TG isoform may also explain the reported absence of TG2 antibodies in a proportion of CD patients and is supported by our finding of high serum titers for anti-TG3 and anti-TG6 IgA, respectively, in two such patients.

### Example 5

### Discrete Antibody Populations in Sera React with TG6 or TG2

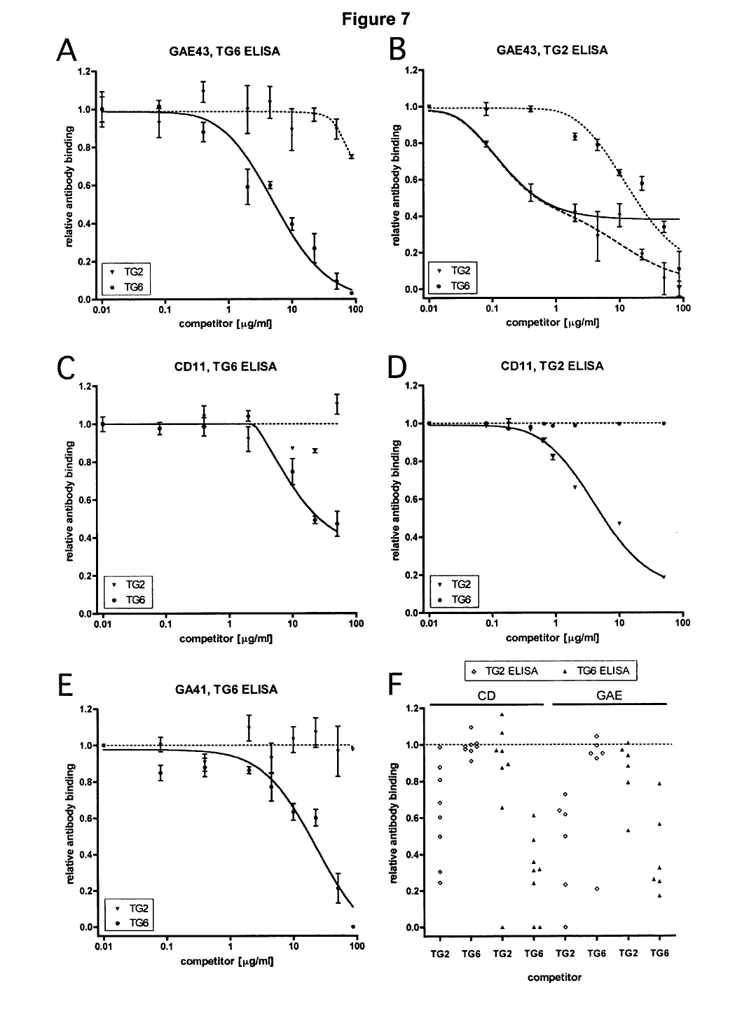

To assess whether isoform-specific antibodies were present or the same antibodies cross-reacted between transglutaminase isozymes, further inhibition studies were carried out on the patient group of Example 4. Sera were preincubated with different concentrations of either the antigen or another TG prior to analysis in the ELISAs. The results are presented as degree of inhibition produced in the ELISA by preincubation with TGs as compared to a control sample preincubated with buffer alone. Representative examples of individual sera are shown in panels A-E of FIG. 7 whereas a comparative analysis with a set concentration of inhibitor is shown in panel F for all patients which displayed reactivity towards both, TG2 and TG6. In most sera, no cross-reacting antibodies could be detected even at high concentrations of inhibitor (FIG. 7C-E). In the anti-TG2 IgA ELISA, TG2 was an effective inhibitor yielding a mean inhibition of 37% (CD) and 55% (GAE) as opposed to 1% (CD) and 15% (GAE) with TG6 (FIG. 7F). Only in 1 (GAE) of 14 patients could significant inhibition by TG6 be detected and therefore, could the presence of antibodies reacting with both isoforms in addition to TG2-specific antibodies be demonstrated (FIG. 7B). Conversely, in the anti-TG6 IgA ELISA, TG6 was the effective inhibitor with a mean inhibition of 71% (CD) and 61% (GAE) in comparison to 18% (CD) and 14% (GAE) with TG2 (FIG. 7F). While with TG2 partial inhibition was seen in 3 sera at much higher concentrations than with TG6, only for 1 patient (CD) was TG2 equally effective as TG6 in blocking the reaction. Despite small sample numbers, a comparison of the inhibition by TG2 or TG6 in the ELISAs (FIG. 7F) showed that the medians differed significantly (p<0.016 for CD, p=0.031 for GAE). These data together with the finding that a number of patients tested positive exclusively for anti-TG6 provide evidence that patients develop populations of antibodies which are specific for or have greater avidity for TG6 than other isozymes.

Thus, not only antibody prevalence (Table 3) but also titers (FIG. 7F) suggest a bias of the immune response towards TG6 in gluten ataxia as opposed to TG2 in CD. In those sera which reacted with both antigens, a higher concentration of TG6 was required for blocking reactivity of sera from GAE than from CD patients in the TG6 ELISA while the opposite was true for TG2 concentrations in the TG2 ELISA (FIG. 7F).

### Example 6

### IgA Deposits in Cerebellum of GA Patient Contain TG6

Post mortem analysis of a GA patientrevealed the accumulation of IgA deposits in the cerebellum and brain stem, most prominently within the muscular layer surrounding vessels but also in brain tissue proper. We have stained frozen sections from various areas of the brain of the same patient using antibodies to TG6 and found co-distribution of TG6 with these IgA deposits. In the cerebellum and medulla, the perivascular areas where an endomysium-associated IgA deposition occurs were intensely positive for TG6 and to a lesser extent brain tissue itself was also stained while staining was absent in the parietal lobe. In contrast, TG6 could not be detected in vascular structures of normal cerebellum.

Variations in the specificity of the antibodies produced in individual patients, from selectivity for a particular TG2 conformation to crossreactivity between TG isozymes, could explain a wide spectrum of manifestations. However, most patients with gluten sensitivity were shown to have antibodies targeting multiple epitopes of TG2 (Sblattero et al., 2002. Eur J Biochem 269:5175-5181). and considering protein homology alone, one would expect to find antibodies crossreacting with further TG isozymes including TG5 and TG7 but thus far we have not been able to identify such antibodies. It is also surprising that in gluten ataxia and dermatitis herpetiformis IgA deposits accumulate in the periphery of vessels in a locale in the tissue where in health TG6 or TG3, respectively, are absent but become abundant in disease (Sardy et al., 2002. J Exp Med 195:747-757; Hadjivassiliou et al., 2006. Neurology 66:373-377). This could indicate that either the deposits originate from immune complexes formed in the circulation or that TG6/TG3 is derived from or its synthesis induced by infiltrating inflammatory cells prior to deposit formation. It is at present unclear whether TG2 and the gluten peptides intersect prior to uptake by antigen presenting cells (APC) or deamidation occurs at the cell surface or in the endocytosis pathway of APCs. The lack of antibodies crossreactive with different TG isozymes in most patients as well as the identification of patients with a response exclusively directed to TG6 or TG3 make epitope spreading less likely the cause for immune responses to other TGs and strongly points to the possibility that TG isozymes other than TG2 can be the primary target of an immune response. However, gluten-dependence of the disease and antibody production implicates the small intestine as the origin independent of subsequent clinical manifestation (Pellecchia et al., 1999. Neurology 53:1606-1608; Hadjivassiliou et al., 2003. J Neurol Neurosurg Psychiatry 74:1221-1224). Unlike TG2 which is expressed in many cell types in the intestinal environment, TG3 and TG6 are essentially absent from the small intestine in health. However, staining of sections from patient biopsies revealed abundant TG6 expression in mucosal APCs in a subset of patients. Furthermore, initial experiments showed that TG6 can deamidate gluten peptides representing common gluten T-cell epitopes. Together with the high degree of isozyme specificity of autoantibodies (FIG. 7), these data suggest that the development of the autoimmune response to TG6 occurs independent of that to TG2 and likely centres on lamina propria macrophages or dendritic cells.

In conclusion, we have shown that antibodies against TG6 can serve as a marker in addition to HLA type, EMA test, and detection of anti-gliadin, anti-deamidated gliadin and anti-TG2 antibodies to identify a subgroup of patients with gluten sensitivity. While anti-TG IgA response is linked with gastrointestinal disease, an anti-TG IgG response is prevalent in gluten sensitive ataxia independent of intestinal involvement but such a response is absent in ataxia of defined genetic origin or healthy individuals. Consequently, we provide a marker that aids the identification of patients with autoimmune-based neurological dysfunction. A number of methods are commonly used for the diagnostic detection of antibodies in samples of body fluids or in tissue samples; examples for such methods include: EIA/ELISA, LiA, FiA, RIA, IRMA, IEMA/EIA, ILMA, IFMA, immunodiffusion, Western-blot, Dot-blot, Immunohistochemistry, protein chip or protein array. Any diagnostic method and device that is suitable for the detection of antibodies or proteins could be adapted for diagnostic purposes following the method described herein in detail by a person skilled in the art.

1. Use of transglutaminase 6, or an antigenically active fragment thereof, for the detection of autoantibodies reacting with transglutaminase 6 for the diagnosis of an autoimmune disorder characterized by the presence of autoantibodies reacting with transglutaminase 6, wherein the autoimmune disorder is a neurological disorder or is characterized by neurological dysfunction.

2. Use according to claim 1, wherein the autoimmune disorder is a paraneoplastic neurological syndrome.

3. Use according to claim 1, wherein the autoimmune disorder characterized by neurological dysfunction is anxiety, depression, brainstem encephalitis, cerebral vasculitis, chorea, dementia, epilepsy, cerebral calcifications, headache with white matter abnormalities, neuromyotonia, myasthenia gravis, myopathy, peripheral neuropathy, a paraneoplastic syndrome, progressive multifocal leukoencephalopathy, progressive myoclonic encephalopathy, schizophreniform disorder or stiff-person syndrome.

4. Use according to claim 1, wherein the autoimmune disorder characterized by neurological dysfunction is ataxia, ataxia with myoclonus, encephalitis, polymyositis, epilepsy with occipital cerebral calcifications, peripheral neuropathy, loss of sensation or muscle weakness.

5. Use according to claim 4, wherein the paraneoplastic neurological syndrome is paraneoplastic cerebellar degeneration, paraneoplastic encephalomyelitis, paraneoplastic opsoclonus-myoclonus, cancer associated retinopathy, paraneoplastic stiff-man syndrome, paraneoplastic necrotizing myelopathy, a motor neuron syndrome including amyotrophic lateral sclerosis (ALS) and subacute motor neuronopathy, subacute sensory neuronopathy, autonomic neuropathy, acute sensorimotor neuropathy, polyradiculoneuropathy (Guillain-Barré), brachial neuritis, chronic sensorimotor neuropathy, a sensorimotor neuropathy associated with plasma cell dyscrasias, vasculitic neuropathy or neuromyotonia.

6. Use according to claim 1, wherein the detection of transglutaminase 6 autoantibodies is combined with the detection of autoantibodies to one or more further transglutaminase isoforms.

7. Use according to claim 6, wherein the one or more further transglutaminase isoforms is selected from transglutaminase 1, transglutaminase 2, transglutaminase 3, transglutaminase 4, transglutaminase 5, transglutaminase 7, coagulation factor XIII or erythrocyte membrane protein band 4.2.

8. Use according to claim 1 wherein the transglutaminase 6 is Ca²⁺-bound activated transglutaminase 6.

9. Use of transglutaminase 6 and transglutaminase 2, or antigenically active fragments thereof, for the detection of autoantibodies reacting with transglutaminase 6 and transglutaminase 2 for the diagnosis of an autoimmune disorder characterized by the presence of autoantibodies reacting with transglutaminase 6 and the absence of autoantibodies to transglutaminase 2.

10. Use according to claim 9 wherein the autoimmune disorder is a cereal protein sensitivity disorder.

11. Use according to claim 10 wherein the cereal protein sensitivity disorder is coeliac disease.

12. Use according to claim 9 wherein the transglutaminase 6 is Ca²⁺-bound activated transglutaminase 6.

13. A kit for diagnosis of an autoimmune neurological disorder characterized by the presence of autoantibodies reacting with transglutaminase 6, the kit comprising transglutaminase 6 or an antigenically active fragment thereof.

14. Use of a kit for the diagnosis of an autoimmune neurological disorder characterized by the presence of autoantibodies reacting with transglutaminase 6, the kit comprising transglutaminase 6 or an antigenically active fragment thereof.
